(19) United States
(12) Patent Application Publication
(10) Pub. No.: US 2020/0378943 A1
(43) Pub. Date: Dec. 3, 2020

(54) Systems And Methods For Providing Micro-Utility Water And Energy Services

(71) Applicant: Cambrian Innovation, Inc., Watertown, MA (US)

(72) Inventors: Justin Buck, Auburndale, MA (US); Matthew Silver, Cambridge, MA (US); William Dean, Watertown, MA (US)

(73) Assignee: Cambrian Innovation, Inc., Watertown, MA (US)

(21) Appl. No.: 16/889,789

(22) Filed: Jun. 1, 2020

Related U.S. Application Data
(60) Provisional application No. 62/854,942, filed on May 30, 2019.

(51) Int. Cl. G01N 33/18; G01N 11/02; G01N 27/416

(52) U.S. Cl. CPC G01N 33/18; G01N 27/4166; G01N 27/4161; G01N 11/02

(57) ABSTRACT

The effectiveness and efficiency of wastewater treatments is computed on a real-time basis

### RELATED APPLICATIONS

The present application claims the benefit of priority from U.S. Provisional Application 62/854,952 filed May 30, 2019 (the “'952 Application”) and is related to U.S. patent application Ser. No. 13/378,763 (“'763 Application”), Ser. No. 13/514,817 (“'817 Application”), Ser. No. 13/811,132 (“'132 Application”), Ser. No. 13/811,149 (“'149 Application”), Ser. No. 13/880,401(“'401 Application), Ser. No. 14/126,264 (“'264 Application”), Ser. No. 14/418,516 (“'516 Application”), Ser. No. 14/526,212 (“'212 Application”), Ser. No. 14/551,462 (“'462 Application”), Ser. No. 14/694,082 (“'082 Application”), Ser. No. 15/691,896 (“'896 Application”), Ser. No. 15/715,801 (“'801 Application”), Ser. No. 16/098,161 (“'161 Application”), and the Ser. No. 16/153,722 (“'722 Application”). The present application incorporates by reference the entireties of the disclosures of all of the above applications including the '952, '763, '817, '132, '149, '264, '516, '212, '462, '082, '896, '801, '161 and '722 Applications as if set forth in full herein.

### INTRODUCTION

There is a need to provide distributed water treatment services to industrial, commercial, and residential entities, such as manufacturing companies, food and beverage companies, commercial office and retail facilities, and residential communities. Such services can include: wastewater treatment services, water supply services, energy (e.g. electricity, heat) generation services, and services involved in environmental sustainability (e.g. carbon credits).

Such services are desirable in order to improve the operation, profitability, sustainability and/or licensing of facilities used to provide services to their end-users and customers.

With respect to energy generation, the ability to generate energy from wastewater and other industrial wastes is believed to be highly desirable because, for example, it helps to offset the cost of treating such wastewater by allowing the operator of a wastewater treatment facility to operate the facility using the energy generated by such treatment. Energy may be generated from wastewater using anaerobic wastewater treatment processes described, for example, in the '763, '896, '132, '462, '161 and '722 Applications assigned to the same assignee as the instant application.

In addition to energy generation, certain sustainability regulations and/or goals require the onsite reuse of water, and encourage the reduction in carbon emissions (i.e., carbon credits). To produce reusable water requires treatment of wastewater. From site-to-site, or even at the same site, the quality (grade) of the reused water needed to be produced may vary depending on regulatory standards or based on the intended application for which the water will be used. Relatedly, because the quality of the water may vary, so too may the treatment process vary. Said another way, to meet a certain water quality standard certain treatment processes may be required. Accordingly, the complexity and cost of a treatment process may be related to the quality of reused water desired or required to be produced.

Monitoring the operation of, and costs associated with, the generation of energy in anaerobic wastewater treatment processes, and the production of reusable water is also desirable in order to insure the short-term and long-term commercial viability of such energy generation and water production.

Accordingly, it is desirable to provide systems, devices and related methods for monitoring the operation of, and costs associated with, the generation of energy and the production of reusable water, among other things.

As a part of a monitoring service there is a need to provide contaminant load measurements (such as biochemical oxygen demand [BOD] or chemical oxygen demand [COD] measurements).

However, the measurement of the concentration of a contaminant (e.g. BOD, COD, and nitrogen) is a non-trivial task. For example, most existing methods require physical sampling of a wastewater stream on a periodic basis. After a sample is taken, the sample may then (typically) be sent to an offsite laboratory for analysis and computation of a BOD or COD concentration based on standard, approved methods, such as those described by the “Standard Methods for the Examination of Water and Wastewater” (ISBN 9780875532875) and “Methods for Chemical Analysis of Water and Wastes” (EPA 600/4-79-020). However, such techniques are too slow to be useful in computing real-time or near-real time measurements of BOD or COD concentration, and, therefore, are not optimal for load or removal rate computations, particularly those needed to make system level operational decisions or determine compliance with operational requirements. Further, such measured concentrations are not (typically) stored electronically by the user who has requested the measurements. Thus, such measurements are unavailable for further analysis and use in a timely manner.

Several methods exist for estimating BOD or COD concentrations from a proxy measurement (i.e., pBOD, pCOD). One of these methods uses an electro-optical device to measure the different constituents present in a wastewater stream as the stream passes through the emitted beam of such a device. Such a device, while providing a quicker turnaround time to estimate BOD concentrations, is expensive to purchase and requires consistent maintenance and calibration to ensure that its' computations remain valid. In sum, its operation is complex and requires substantial investment in equipment and personnel training. In addition, by relying on optical methods based on physiochemical properties, these methods do not directly assess the biological or chemical reactivity of the constituents in the water, which introduces potential errors in the representative estimation of BOD and COD concentrations.

Accordingly, it is desirable to provide systems and methods for estimating COD and BOD loads based on COD and BOD concentrations on a real-time or near real-time basis that overcome the disadvantages of existing methods.

### BRIEF DESCRIPTION OF DRAWINGS

FIG. 1 depicts a simplified block diagram of an exemplary system 1 for monitoring one or more industrial wastewater treatments according to an embodiment of the invention.

FIG. 2 depicts a simplified block diagram of an exemplary computational and analysis section according to an embodiment of the invention.

FIG. 3 depicts an exemplary graph of BOD concentration data in accordance with one or more embodiments of the invention.

FIG. 4A depicts exemplary graphs of a plurality of methods for computing BOD concentrations from BOD data samples according to embodiments of the invention.

FIG. 4B depicts an exemplary graph of values of an average flow computed based on measured and sampled wastewater flow data in accordance with an embodiment of the invention.

FIG. 4C depicts an exemplary graph of BOD concentration values computed using an exemplary period-average approximation method in accordance with an embodiment of the invention.

FIG. 4D depicts an exemplary graph of a computed BOD concentration, a computed average wastewater flow and a computed BOD load in accordance with embodiments of the invention.

FIG. 4E depicts exemplary graphs of BOD concentration computed by using an exemplary midpoint approximation method in accordance with an embodiment of the invention.

FIG. 4F depicts an exemplary graph of a computed BOD concentration, computed average wastewater flow and computed BOD load in accordance with still another embodiment of the invention.

FIG. 4G depicts an exemplary graph of BOD concentration values computed by using an exemplary linear interpolation approximation method in accordance with an embodiment of the invention.

FIG. 4H depicts an exemplary graph of a computed BOD concentration, computed average wastewater flow and computed BOD load in accordance with yet another embodiment of the invention.

FIG. 4I depicts exemplary graphical representations of daily BOD load computations in accordance with embodiments of the invention.

FIG. 5A depicts exemplary graphical representations of wastewater flow values according to embodiments of the invention.

FIG. 5B depicts exemplary graphical representations of computed daily flow data, computed average flow data and computed peak flow data based on a weekly reporting time period or window according to embodiments of the invention.

FIG. 5C depicts exemplary graphical representations of computed BOD concentration values according to embodiments of the invention.

FIG. 5D depicts an exemplary graphical representation of computed daily BOD concentrations, computed average BOD concentrations and computed peak BOD concentrations based on a weekly reporting time period or window in accordance with embodiments of the invention.

FIG. 5E depicts exemplary graphical representations of computed BOD load values in accordance with embodiments of the invention.

FIG. 5F depicts graphical representations of computed daily BOD loads, computed average BOD loads and computed peak BOD loads based on a weekly reporting time period or window in accordance with embodiments of the invention.

### SUMMARY

The inventors provide a number of different systems and methods for monitoring wastewater, among them is an exemplary method comprising: receiving one or more measurements from one or more sensors or analytical instruments and one or more flow meters over a selectable length of time at a controller (e.g., a programmable logic controller), and computing a contaminant load (e.g., BOD, COD, pBOD (BOD proxy), or pCOD (COD proxy)) of the wastewater based on the received measurements. The measurements from the one or more sensors, analytical instruments and the one or more flow meters may be received during start-up of a wastewater system, and/or, alternatively, during operation of the wastewater system.

Such an exemplary method may further comprise sampling the measurements from the one or more flow meters instantaneously (e.g., every 1 to 1000 milliseconds), and sampling the measurements from the one or more sensors or analytical instruments periodically (e.g., every day), where the sensors and or analytical instruments are not sampled as often as the flow meters during a same time period.

In an embodiment, the one or more sensors may comprise one or more BOD sensors for completing pBOD proxy measurements. Yet further, the one or more BOD sensors may comprise one or more electrodes of a bio-electrochemical system, and the exemplary method may then further comprise measuring an electrical current or voltage generated by the one or more electrodes; communicating electronic signals representing the measured current or voltage measurements and a sampled wastewater flow from the one or more flow meters to the controller or another controller; converting the electronic signals representing the measured current or voltage measurements into a BOD concentration; and computing the BOD load based on the computed BOD concentration and the sampled wastewater flow.

The same or a related method may comprise adjusting data from the sampling of the one or more flow meters and data from the sampling of the one or more sensors or analytical instruments so that the time period over which both sets of data are sampled is the same.

Yet further, the exemplary method may comprise computing one or more resource-related values or amounts (e.g., fees, rates, a billing rate) derived from measurements received from one or more flow meters and from one or more sensors or analytical instruments. The resource-related values or amounts may also represent a combination of a fixed or variable value or values derived from measurements received from one or more BOD sensors and one or more flow meters or analytical instruments.

Further, the computed one or more resource-related values or amounts (e.g., fees, rates) may be derived from data samples associated with one or more of (i) measuring a total volume of the wastewater treated, (ii) measuring a volume of the wastewater treated over a set time period (i.e., flow rate), (iii) characteristics of the wastewater being treated, including, but not limited to, the amount of a contaminant in influent wastewater, (iv) characteristics of the wastewater being discharged, including, but not limited to, the amount of a contaminant in effluent wastewater, (v) a computation of the amount of contaminants removed from the wastewater or (vi) a direct measurement of a current or voltage of one or more biologically-catalyzed electrodes.

In addition to the features described above, an exemplary method may yet further comprise at least one of the following: treating a volume of the wastewater over a set time period, and (ii) measuring an amount of contaminants contained in influent wastewater based on the measurements from the one or more sensors, analytical instruments or flow meters, measuring an amount of contaminant in effluent wastewater based on the measurements from the one or more sensors, analytical instruments or flow meters, and computing an amount of contaminant removed from the wastewater from the measured amount in the influent, the measured amount in the effluent, and a flow rate.

Another exemplary method for monitoring wastewater may comprise measuring a rate of one or more parameters i entering a wastewater treatment system over a plurality of different time intervals (e.g., peak minute, peak hour, peak day, peak week), where the rate may be selected from a peak rate or an average rate of the one or more parameters. Such an exemplary method may further comprise computing a value for a parameter i , comparing the computed value to a threshold for the parameter i; and computing an overage value for the parameter i when the comparison indicates the computed value exceeds the threshold by determining a difference between the computed value and the threshold.

Still further, the exemplary methods may additionally comprise computing a percent removal nᵢ of parameter i from an influent stream of the wastewater by the treatment system, where the percent removal is computed from an average inlet concentration, (C_{i,1},), and an average outlet concentration, (C_{i,2}), where

$${n}_{i} = \frac{{c}_{i , 1} - {c}_{i , 2}}{{c}_{i , 1}} \times 100 \% ,$$

and/or computing an amount of parameter i loaded into, or removed from, the wastewater by generating a sum of the product of a total volumetric flow of wastewater, (V), and a difference in an inlet concentration, (C_{i,t,1}), and an outlet concentration (C_{i,t,2}), over all time intervals for a given time interval t, represented as follows:

(Loading) Sᵢ=ΣₜC_{i,t,1}×Vₜ

(Removal) Sᵢ=Σₜ(C_{i,t,1}−C_{i,t,2})×Vₜ

Yet another exemplary method for may comprise collecting data from a wastewater stream associated with one or more parameters i, where the one or more parameters i are measured in an influent or effluent stream of the wastewater stream, collecting the data over a plurality of different time intervals (e.g. millisecond, second, minute, hour, day, week), determining one or more of (i) a peak value of the one or more parameters, (ii) average value of the one or more parameters, and/or (iii) a total value of the one or more parameters.

Such an exemplary method may further comprise associating a stored, pre-determined resource-related value (e.g., monetary amount) to one or more computations of the one or more parameters i entering the wastewater system over the plurality of different time intervals.

### DETAILED DESCIPTION, WITH EXAMPLES

As used herein, the words “comprising”, and any form thereof such as “comprise” and “comprises”; “having”, and any form thereof such as “have” and “has”; “including”, and any form thereof such as “includes” and “include”; and “containing” and any form thereof such as “contains” and “contain” are inclusive or open-ended and do not exclude additional, unrecited elements or process steps.

As used herein, the term “stream” can include various molecules in liquid, gas, or solid state passed from one location to another, and can include mixtures of gases, liquids, and particulate solids. Generally, an exemplary stream can be a wastewater stream or a biogas stream containing methane.

As depicted, process flow lines in the figures can be referred to interchangeably as, e.g., lines, pipes, feeds, portions, products, or streams.

As used herein, the term “about” or “approximately” is defined as being close to or near as understood by one of ordinary skill in the art, and in some embodiments may be quantified as within 10%, more particularly within 5%, still more particularly within 1%, and is in some cases within 0.5%.

As used herein, the term “a” or “an” when used in conjunction with the term comprising or a form thereof may mean “one”, but is also consistent with the meaning of “one or more”, “at least one”, and “one or more than one”.

As used herein, the term “hour” may be abbreviated “hr”, the term “kilogram” may be abbreviated “kg”, the term “Pascal” may be abbreviated “Pa”, the term “milligram” may be abbreviated “mg”, the term “kilogram” may be abbreviated “kg”, the term “liter” may be abbreviated “L”, the term “meter” can be abbreviated “m”, the phrases “meter-cubed” or “cubic meter” may be abbreviated “m³”, the phrase “gallons per minute” may be abbreviated “gpm”, the phrase “gallons per day” may be abbreviated “gpd”, the phrases “biological oxygen demand” or “biochemical oxygen demand” may be abbreviated “BOD”, the phrase “chemical oxygen demand” may be abbreviated “COD”, the phrase “degrees Celsius” may be abbreviated “° C.”, and the phrase “degrees Fahrenheit” may be abbreviated “° F.”. All pressures are absolute. It should be further understood that when used herein, the term BOD may be used as shorthand to describe any and all of the methods for determining biochemical oxygen demand (e.g. BODS and BODU) and/or a sub-set of biochemical oxygen demand (e.g. tBOD, sBOD, cBOD, etc.). Likewise, the term COD may also be used as shorthand to refer to any and all methods for determining chemical oxygen demand and/or a sub-set of chemical oxygen demand (e.g. tCOD, sCOD, ssCOD, bCOD etc.). The terms BOD and COD may also be used to represent proxy measurements (pBOD, pCOD) that can be correlated to the values obtained by standard analytical methods.

It should also be understood that one or more exemplary embodiments may be described as a process or method. Although a process/method may be described as sequential, it should be understood that such a process/method may be performed in parallel, concurrently or simultaneously. In addition, the order of each step within a process/method may be re-arranged. A process/method may be terminated when completed and may also include additional steps not included in a description of the process/method.

As used herein, the term “and/or” includes any and all combinations or permutations of one or more of the associated listed items.

It should be understood that when used herein, the designations “first”, “second”, “third”, etc., is purely to distinguish one component or part of a process from another and does not indicate an importance, priority or status unless the context, common sense or recognized knowledge of those skilled in the art indicate otherwise. In fact, in some cases the component or parts of a process could be re-designated (i.e., re-numbered) and it would not affect the scope of the present invention.

As used herein the phrases “connection”, “connected to”, or similar phrases mean an indirect or direct physical connection between at least two different parts of a device or system or means one part of a device or system is subsumed within (and thereby connected to) at least one other part of a device or system. It should be understood that when one part of a device or system is described or depicted as being connected to another part, other components used to facilitate such a connection may not be described or depicted because such components are well known to those skilled in the art.

Yet further, when one part of a device or system is described or depicted as being connected to another part using “a connection” (or single line) in a figure it should be understood that practically speaking such a connection (line) may comprise (and many times will comprise) more than one physical connection or channel, may be omni-directional or bi-directional.

It should be noted that the systems and devices, as well as any subsystems, etc., thereof, illustrated in the figures are not drawn to scale, are not representative of an actual shape or size and are not representative of any actual system, platform or device layout, or manufacture's drawing. Rather, the systems and devices are drawn so as to help explain the features, functions and processes of various exemplary embodiments of the present invention described herein.

As used herein the phrases “operable to” and “configured to” mean “functions to”.

As used herein, the terms “embodiment” or “exemplary” refer to an example of the present invention.

It should be understood that when the description herein describes the use of a “programmable logic controller (PLC)”, “controller”, “electronic processor”, “specialized microcomputer” or “processor” that such a device includes stored, specialized instructions for completing the associated, described features and functions (e.g., computing, aggregating, analyzing, approximating, measuring, monitoring, normalizing, retrieving, reporting, sampling, displaying, etc.). Such instructions may be stored in an onboard memory or in separate memory devices. Such instructions are designed to integrate specialized functions and features into the PLC, controller, electronic processor or processor that are used to complete the inventive functions, features, methods and processes described herein by, for example, controlling one or more inventive systems or devices or their constituent elements used in such a method or process. Further, it should be understood that when a PLC, controller, electronic processor or processor executes a set of such stored instructions the executed instructions may constitute steps in an inventive process or in an application. Yet further, it should be understood that each of the embodiments of a PLC, controller, electronic processor or processor described herein process information (e.g., measurements) much faster than humanly possible and exchange information with other devices (e.g., measurement components, other PLCs, controllers, electronic processors or processors) much faster than humanly possible. Accordingly, it should be understood that each of the embodiments of the present invention cannot practically be implemented in any amount of time that would be acceptable to one skilled in the art using human beings as substitutes for the PLCs, controllers, electronic processors or processors described herein. Nor can it be said that such embodiments are well-understood, routine, or conventional because such embodiments are not widely prevalent or in common use in the treatment of water. For example, the embodiments described herein involve the transmission of measured data (measurements) that must be substantially, immediately processed in order to effectively monitor water and energy systems and to take actions in response to such data in real time or near real-time, for example. Accordingly, the speeds at which the measured data and actions, and the amount of measured data and actions, is many times greater than can be communicated and processed by the human mind within the time periods demanded by users of embodiments of the present invention and those skilled in the art of the present invention.

The terms “values”, “data”, “data samples” and “samples” may be used interchangeably herein to mean information related to a measured, monitored, detected or stored characteristic of a portion or sample being measured, such as a portion or sample of wastewater, or related to the operation of inventive systems provided by the present invention.

As used herein the phrases “sampling interval”, “measurement interval”, “sampling frequency” and “measurement frequency” may be used interchangeably and mean the length of time between raw data points (i.e., interval) or the number of data points in a fixed time period (i.e., frequency). For example, data generated by a PLC capable of making millions of data computations (i.e., “rich” data) typically will have a short interval, typically ranging from microseconds to minutes. In comparison, manually sampled data will typically have longer (larger) intervals, and therefore lower frequencies, such as one sample per hour, per day, per week, or per month.

As used herein the phrase “computation period” means a time period over which a data value is computed. During a given computation period a data value or values may be either “filled in” (i.e., added) or averaged to represent values for the computation period. The computation period can be longer or shorter in duration than a sampling interval. If the computation period is longer than the sampling interval (meaning many data samples, sampled during many sampling intervals, may be taken during the period), a single representative data value may be produced from the many data samples using a data reduction method. If, however, a computation period is shorter than a sampling interval (meaning a value may be computed before a sampling period begins), data samples may be “filled in” for the computation period based on an approximation method. An exemplary computational period may be one hour in duration.

As used herein the phrases “summation period” or “summary period” mean a time period over which computed data may be consolidated, aggregated, or summarized for presentation or further analysis. The length (i.e., time) of a summation period can match a computation period or be longer. Data samples or computations sampled or computed during a computation period may be reduced using a data reduction method. An exemplary summation period may be one calendar day in duration.

The phrases “reporting period”, “reporting cycle”, “billing period” or “billing cycle” may be used interchangeably and mean a time period over which summary data may be reported and analyzed. Statistical methods can be applied to data in a reporting period for the purpose, for example, of correlating summarized data values or computed values of technical performance to a bill or invoice for services that have been completed (e.g., amount of water treated). An exemplary reporting period may be one calendar week or one calendar month.

The phrase “data reduction method” means a method or process for creating a smaller amount of data from a more extensive amount of data. Some inventive exemplary methods include, but are not limited to: an averaging method, a weighted average method (e.g. time-weighted, volume-weighted), and a summation method, for example.

The phrase “approximation method” means an inventive method or process for “filling in” (adding) data between sampled data values using a specified method. Some inventive exemplary methods include, but are not limited to: a period average method, a midpoint method, a left-hand method, a right-hand method, a linear interpolation method, a spline fitting method, and a process modeling method, for example.

As used herein the phrase “statistical method” means an inventive method for analyzing data (e.g., a set of data) to report characteristic features of such data. Some inventive exemplary statistical methods include, but are not limited to: a mean (average) method, a median method, a mode method, a maximum method, a minimum method, and a standard deviation method.

It should be understood that the phrases “sensor” and “meter” may be used interchangeably herein unless the context, common sense and/or the knowledge of one skilled in the art dictates otherwise.

Though the principles of the invention may be applied to a number of different applications, for purposes of the explanation that follows we will focus on wastewater treatment, water re-use, and energy generation.

Accordingly, in order to effectively monitor the operation of, and costs associated with, a wastewater treatment facility (that includes the generation of energy and the production of reusable water) requires an understanding of the processes involved.

For example, one wastewater treatment process may require initial start-up procedures in addition to on-going treatment procedures, each being associated with different costs that must be monitored and accounted for. In one embodiment, an inventive system is provided for monitoring initial or start-up procedures and their associated fixed costs (e.g., connection or capacity fees) and on-going treatment procedures and their associated variable costs (e.g., costs per volume, mass, or unit energy). In more detail, the size and complexity of a given wastewater treatment facility varies from location to location, and from application-to-application. So, too do the complexities of such facilities and their associated costs. In general, the larger the facility, and/or the more complex the treatments provided by the facility, the higher the fixed costs will be. This is due, at least in part, to the fact that larger facilities require more physical systems (hardware), which take longer to install and start-up. Similarly, the more complex the treatment process, the longer it will take (generally speaking) to install and start-up the system(s) responsible for completing such complex processes.

The variable costs associated with a wastewater treatment process depends mainly, but not exclusively, on the typical characteristics of the wastewater that needs to be generated by a particular application before treatment (referred to herein as “inlet” or “influent” wastewater”), the typical characteristics of the water that is discharged after treatment but before the treated wastewater is reused in the application or released to the environment (referred to herein as “treated”, “outlet”, “discharged” or “effluent” water), the volume of water processed, and the requirements for reporting and compliance.

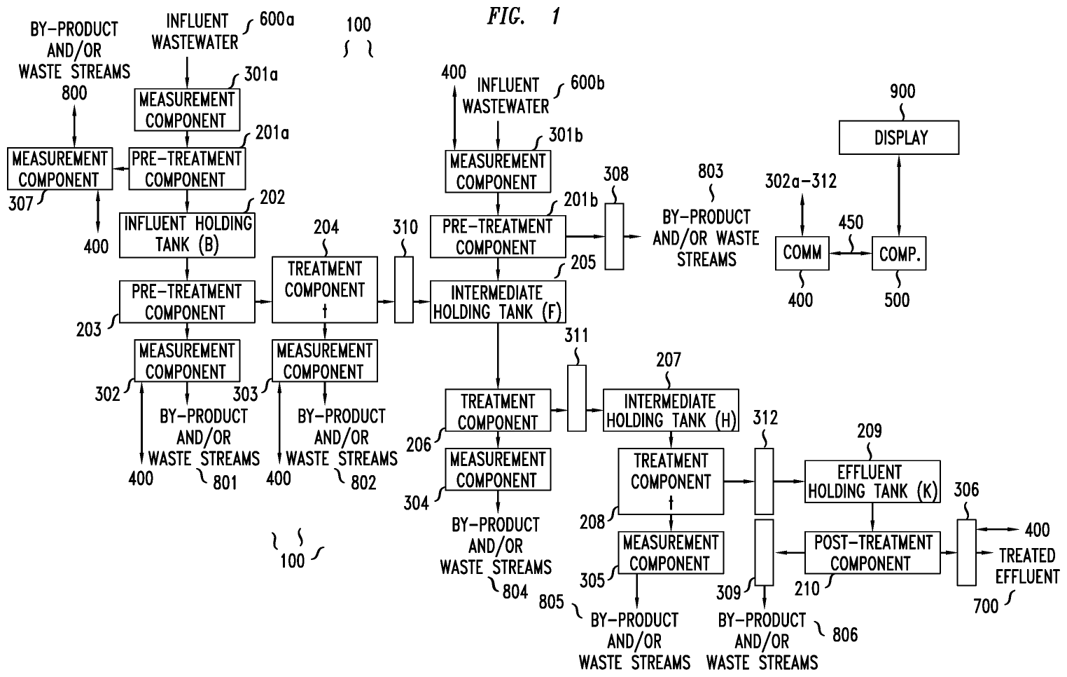

Referring now to FIG. 1, there is depicted a simplified block diagram of an exemplary system 100 for monitoring one or more wastewater treatments. As shown, the system 100 may comprise treatment components 201a to 210, measurement components 301a to 312, communications component 400 and computation component 500.

Collectively, the treatment components 201a-210 may be a part of a system for treating wastewater, such as an anaerobic treatment system described in the '763, '896 and '132 Applications assigned to the same assignee as the instant application. In a further embodiment, the components 201a-210 may also be a part of an anaerobic bio-electrochemical system (BES) that includes one or more electrodes. The treatment components 201a-210 may also comprise, to name just a few exemplary components, aerobic biological treatment components (e.g. membrane bioreactor, activated sludge, moving bed bioreactor), nutrient removal components (e.g. nitrification, denitrification, anammox, biological phosphate removal), pre-treatment components (e.g. pH adjustment, equalization), solids removal components (e.g. clarifier, settler, screen, filter, centrifuge), tertiary treatment components (e.g. reverse osmosis, ion exchange, carbon filtration), or disinfection components (e.g. chlorination, ozonation, UV sterilization).

It should be understood that each component 201a-210 may, depending on the application, comprise more than one element. In more detail, the following is a non-limiting, non-exhaustive list of some exemplary, specific examples of each component 201a-210:

components 201a and 201b: in embodiments these component may comprise means for adding a caustic, acid (to adjust pH), buffer and/or nutrient, for example, including but not limited to a filter, clarifier, vertical lamella clarifier, centrifuge, grinder, macerator, a dosing pump and a mixer;

component 202: in embodiments component 202 may be anaerobic treatment means or denitrification means, including, but not limited to for example, equalization tank, surge tank, calamity tank, hydrolysis tank, acidification tank, pre-fermentation tank;

component 203: rotary drum screen, filter, clarifier, vertical lamella clarifier, centrifuge, grinder, macerator, where in preferred embodiments component 203 may comprise means for removing suspended solids, such as the rotary drum screen or means for adding a caustic, acid (to adjust pH), buffer and/or nutrient, for example;

component 204: in embodiments this component may comprise anaerobic treatment means such as anaerobic treatment reactor, expanded granular sludge bed, upflow anaerobic sludge blanket, internal circulation reactor, anaerobic membrane bio-reactor, anaerobic moving bed bio-reactor, anaerobic fluidized bed, anaerobic digestor, and/or denitrification system;

component 205: break tank, surge tank, calamity tank, equalization tank, holding tank, mixing tank, settling tank, pre-aeration tank;

component 206: in embodiments this component may comprise aerobic treatment means including, but not limited to, an aerobic reactor (e.g., membrane bioreactor, activated sludge process, trickling filter, moving bed bio-reactor, aeration lagoon), nitrification system, and/or denitrification system;

component 207: break tank, surge tank, equalization tank, holding tank, settling tank, aeration tank;

component 208: in embodiments this component may comprise means for removing dissolved solids, such as a suspended solids removal system (e.g. microfiltration system, ultrafiltration system, clarifier), dissolved solid/ion removal system (e.g., reverse osmosis unit, nanofiltration system, electrodialysis system, electrodialysis reversal system, ion exchange system, forward osmosis system, coagulation/flocculation/filtration), aerobic treatment system, advanced oxidation or disinfection systems (e.g. UV disinfection system, ozonation system, system, plasma system).

component 209: reuse water tank, clear well, buffer tank, contact tank; and

component 210: in embodiments this component may comprise means for disinfecting water or means for adding chlorine or chloramine to water, such as: a disinfection system (e.g. UV disinfection system, ozonation system, chemical disinfection (e.g. chlorine, chloramine) system), dissolved solid/ion removal system (e.g. reverse osmosis system, forward osmosis system, nanofiltration system, coagulation/flocculation system), suspended solids removal system (e.g. clarifier, microfiltration system, ultrafiltration system, advanced oxidation system (e.g. plasma system), where one preferred component comprises a disinfection system that comprises means for adding chlorine or chloramine to water.

Typically, the characteristics of inlet wastewater 600a, 600b being treated by the treatment components 201a-210 and the costs associated with treating the wastewater 600a,b (e.g., costs associated with the operation of the system), may be substantially dependent upon several key parameters, most notably contaminant (e.g. BOD, COD) concentrations and relative composition of organics in the inlet wastewater 600a, 600b. In general, the higher the contaminant concentration and or the more complex the compositions making up the BOD, COD and organics the more complex the system and its processes required to effectively treat the inlet wastewater 600a,b.

In addition, the composition of any solids (e.g. as represented by total suspended solids (TSS), total dissolved solids (TDS), volatile suspended solids (VSS), etc . . . ), and the level of any nutrients (e.g., nitrogen, phosphorus, and sulfur), along with the pH and temperature of the inlet wastewater 600a,b may affect the overall characteristics of the inlet wastewater 600a,b which in turn may dictate or affect the type of treatment component 201a-210 and related processes required to produce reusable or dischargeable wastewater 700. Collectively, the combination of any constituents of the wastewater stream targeted to be removed may be referred to as contaminants. Such contaminants may typically include BOD and COD, but may also include, amongst others, solids, inorganic salts, nutrients (such as nitrogen and phosphorus species), hydrogen ions, hydroxide ions, and thermal energy generating constituents. Further, when referred to herein, a contaminant may refer to one or more constituents or one or more characteristics of a constituent of an influent stream, e.g., streams 600a,b, that is required or desired to be altered before discharge in an effluent stream, e.g., stream 700.

In embodiments of the invention, one or more measurements may be received by a PLC from one or more sensors or analytical instruments and one or more flow meters over a selectable length of time at a controller. Thereafter, the PLC may be operable to compute a contaminant load of the wastewater based on the received measurements.

Further, one or more exemplary measurements may be made by components 301a to 312 at (i) an inlet of the system 100 (e.g., by components 301a, 301b) (ii) within the system 100 (e.g., by components 310-312), (iii) at an outlet or discharge position of the system (e.g., by components 302 to 309), and/or (iv) at some combination of inlet, within, outlet and discharge positions of the system 100. For clarity, measurement components are shown on a stream (inlet, outlet, or connecting treatment process), but may be located on or within the treatment components (201a-210), or other locations within the system 100. A decision as to where to position a measurement component and what type of measurements should, or must, be made by a measurement component at a particular position may involve consideration of the treatment component(s) 201a-210 involved and/or regulations governing discharges, for example.

In one embodiment, one or more of the measurement components 301a to 312 may comprise one or more in-line monitoring equipment (e.g., sensors) positioned in, or in contact with, a wastewater stream. Such sensors and/or analytical instruments may be operable to measure the levels of: (i) one or more parameters or properties, (ii) a contaminant, such as the BOD concentration or COD concentration, (iii) inlet wastewater 600a,b the (iv) effluent or discharge wastewater 700, or (v) some parameter associated with a treatment component 201a-210, for example.

The measurement components 301a-312 may further comprise one or more analog or digital measurement devices, such as flow meters, where each flow meter may be operable to measure the flow (volume per time period) of inlet wastewater 600a,b, flow through a treatment component 201a to 210, and flow of effluent or discharged wastewater 700, for example. Still further, additional examples of exemplary measurement components 301a to 312 may include, but are not limited to: one or more analytical instruments, analog or digital electrochemical analyzers, optical analyzers, mechanical, electrical, and thermal analyzers, including, but not limited to spectrum analyzers and impedance analyzers. In addition, a component 301a-312 may comprise an electricity meter for measuring the amount of electricity used, and generated, by a component 201a-210 and by the overall system 100.

Still further, one or more of the measurement components 301a to 312 may comprise a controller, such as a PLC operable to receive signals from one or more sensors, analytical instruments, and/or flow meter(s),to name just a few of the devices a PLC may receive signals from. For example, a PLC may be operable to receive signals from sensors, analytical instruments and flow meters that are used to sample and/or measure characteristics or properties of the inlet wastewater 600a,b (and effluent or discharged wastewater 700) over a period of time (i.e., a sampling interval, e.g., day, week, monthly). In an embodiment, one or more PLCs may be operable to alter the operating conditions of the system 100, including any of the operating parameters or inputs of the treatment components 201a-210. A PLC may access stored instructions to complete one or more processes or methods for (i) optimizing treatment rates or throughputs, (ii) optimizing resources (e.g. electricity or chemical), or (iii) managing the operation of the system 100, for example, in accordance with the physical, desired or expected limits of the system 100 as agreed to by an operator of system 100.

It should be further understood that each component 301a-312 may comprise analog or digital elements/circuitry and, depending on the application, measure and/or generate analog and/or digital signals. In addition, each component 301a-312 may, depending on the application, comprise more than one element. In more detail, the following is a non-limiting, non-exhaustive list of some exemplary, specific examples of each component 301a-312:

components 301a to 302, 307, 308: flow meter, BOD sensor, pH sensor, COD sensor, TSS sensor, temperature sensor, other nutrient/contaminant sensors;

component 303: biogas flow meter, lower explosive limit sensor, methane fraction sensor, H₂S sensor, pressure sensor, temperature sensor, relative humidity sensor, oxygen sensor;

component 304: flow meter, TSS sensor, turbidity sensor, gas flow meter, nutrient/contaminant sensor, TDS sensor;

component 305: TDS sensor, flow meter, pH sensor, temperature sensor, conductivity sensor;

component 306: flow meter, TDS sensor, chlorine sensor, turbidity sensor, TOC meter, ozone sensor, TSS sensor;

component 309: flow meter, TSS sensor, temperature sensor, BOD sensor, COD sensor, other nutrient/contaminant sensors, gas flow meter, gas composition sensor;

components 310 and 311: flow meter, BOD sensor, temperature sensor, pH sensor, COD sensor, other nutrient/contaminant sensors, TDS sensor, TSS sensor, conductivity sensor; and

component 312: flow meter, TDS sensor, temperature sensor, pH sensor, BOD sensor, COD sensor, TSS sensor, conductivity sensor, other nutrient/contaminant sensors.

In accordance with embodiments of the invention, one or more of the measurement components 301a to 312 (e.g., all of them) may be connected via wired or wireless means known in the art (not shown in figures) to the communications component 400. Further, one or more of the measurement components 301a to 312 (e.g., a PLC) may be operable to convert signals representative of raw, measured data to one or more appropriate wired or wireless signals (e.g., Ethernet, Bluetooth, 802.11, 4G LTE, 5G NR, NB-IoT, eMTC, EtherNet/IP, DeviceNet, ControlNet, Optomux, Modbus, Profibus, PROFINET, HART, etc.) and transmit such signals to the communication component 400. Upon receipt of such signals the communications component 400 may be operable to, in turn, transmit signals to computation component 500 via a wired or wireless communications path 450 for example. In embodiments, the computation component 500 may be co-located with the treatment components 201a to 210 or may be remote from the treatment components 201a to 210. In an embodiment, the communications component 400 may include transceiving circuitry for converting and formatting data representative of a sampled measurement received from a measurement component 301a to 312 into signals that can be transmitted to the component 500 via path 450 which may be a part of a public telecommunications network or private local network, for example, where the component 500 may include its own transceiving circuitry for receiving such transmitted signals. Further, communications component 400 may be operable to transmit signals it receives from computation component 500 to the treatment components 201a to 210 or measurement components 301a to 312 for adjusting the operation of one or more of such components.

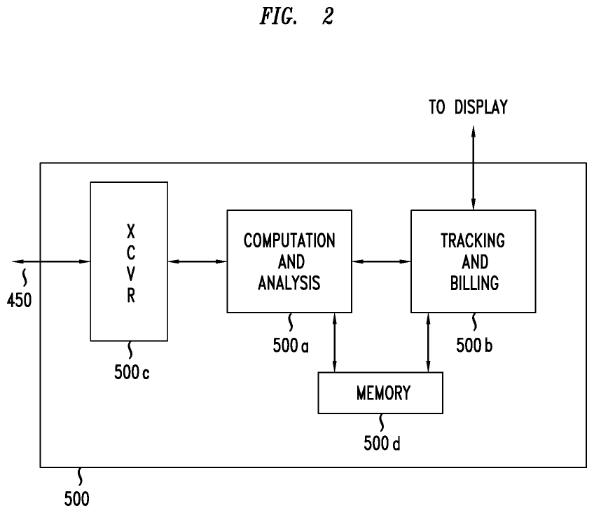

Referring now to FIG. 2, there is shown an embodiment of a computation component 500. As shown, the component 500 may comprise transceiving circuitry 500c and a computation and analysis section 500a. In an embodiment, the computation and analysis section 500a may comprise a controller operable to receive and store signals received from the communications component 400 (or, alternatively, directly from one or more measurement components 301a to 312), and compute real-time, instantaneous values or, alternatively, compute statistical or average values, such as utilization values. In embodiments of the invention, the utilization of on-site water and energy are two examples of such utilization values that may be computed by component 500.

For example, component 500a may be operable to analyze and compute an on-site water utilization value based on signals derived from data representative of volume or flow measurements made by one or more measurement components 301a to 312 (e.g., a flow meter, sensors), or based on the measurement of the amount of TDS (e.g. via conductivity measurements) in the output of treatment component 208 (e.g., a reverse osmosis membrane filter). In addition, section 500a may be operable to compute energy utilization values based on signals derived from one or more of the measurement components 301a-312. One or more of the measurement components 301a-312 may comprise, for example, one or more energy and/or electricity meters operable to measure the energy used or generated by a treatment component 201a-210 and/or the electricity used or generated by a component 201a-210, for example. Data representative of such measurements may be transmitted to the component 500 via component 400 over path 450 for analysis and computation of additional values.

In one embodiment the computation and analysis section 500a may be operable to (a) receive signals from a measurement component 301a to 312, (b) compute energy and/or water usage values, for example, based on the received signals, (c) retrieve historical or pre-determined values (e.g., thresholds, statistical data) or water and/or energy usage from electronic memory (e.g., a database, on-board memory circuitry, electronic chips) and (d) compare such retrieved values to the computed values in order to (e) generate an indicator that indicates (i) whether the system 100 is using more, the same, or less water and/or energy than used previously, and/or (ii) whether the system 100 is using water and/or energy that exceeds a pre-determined threshold value(s), for example.

In addition to computing water and energy utilization, component 500 may be operable to compute the amount and type of nutrients (e.g. nitrates, ammonias, phosphates, sulfates) within inlet and discharge wastewater streams 600a, 600b, 700 based on signals representative of measurement samples of such wastewater received from one or more measurement components 301a-312 (e.g. as described in the '817, '082 Applications) via communications component 400 and path 450. Yet further, the computation component 500 may be operable to compute nutrient-related values, for example, based on the received signals, retrieve historical, statistical or pre-determined nutrient-related (e.g., thresholds) values from electronic memory (e.g., a database, on-board memory circuitry, electronic chips) and compare such retrieved values to the computed values in order to generate an indicator that indicates (i) the type and amount of nutrients in inlet wastewater 600a,b, (ii) the type and amount of nutrients in discharge wastewater 700, (iii) the type and amount of nutrients removed or added by one or more treatment components 201a-210, (iv) one or more ratios based on the indicators in (i) through (iii) immediately above, and (v) whether a nutrient or nutrients in inlet or outlet discharge wastewater 600a, 600b, 700 exceeds a pre-determined threshold value(s), for example.

Once again, to complete the computations and analyses described herein the computation and analysis section 500a may comprise a controller operable to store instructions in the form of electrical signals in an on-board or associated memory 500d, store referential, historical and/or statistical data, and then retrieve and/or execute the instructions along with stored received signals and any referential, historical or statistical data in order to complete a particular computation or analysis. The instructions may comprise one or more steps or processes that, when combined, make up a particular computation or analysis. For example, one set of instructions may form a particular averaging analysis and computation, while another set of instructions may comprise a comparison of signals or data associated with the same, or a different, measurement parameter, water characteristic or property, for example.

More particularly, the computation and analysis section 500a may be operable to complete a plurality of computations and analyses (i.e., functions) based on signals (sampled data) received from components 301a-312, among them: (i) compute and analyze the chemical, biological and physical constituents of inlet and outlet wastewater 600a, 600b, 700 (ii) compute the instantaneous BOD or COD load of inlet wastewater 600a, 600b, (iii) compute the instantaneous BOD or COD level of discharged, treated wastewater 700 (iv) compute other parameters related to COD and BOD (e.g. tCOD, tBOD, sCOD, sBOD, bCOD, cBOD, etc.) (v) compute the amount and concentration of nutrient constituents (e.g. nitrogens (total nitrogen, TKN, nitrates, nitrites, ammonias), phosphates (orthophosphates, total phosphorous), sulfates (sulfate, total sulfur) in the wastewater; (vi) compute the flow of inlet wastewater 600a, 600b (volume per unit of time); (vii) compute the flow of effluent or discharged water 700; (viii) compute TDS (e.g. based on conductivity measurements), TS, TSS, VSS, pH, temperature, conductivity, or other characteristics; (ix) compute energy usage; and (x) compute energy generation, among other computations.

Yet further, the computation component 500 may be operable to transmit configuration signals, among other signals, to one or more of the measurement components 301a-312 or treatment components 201a-210 via the communications component 400 over path 450 in order to configure the one or more measurement components 301a-312 or treatment components 201a-210 to control their operation (e.g., sensors, flow meters, etc.).

Referring back to FIG. 1, as mentioned previously the measurement components 301a-312 may include one or more BOD sensors, each operable to measure an electrical current generated by an electrode of a bio-electrochemical system (BES), for example. In an embodiment, the measured current (or voltage) is representative of a BOD concentration (e.g., proportional to a concentration).

Further, the measurement components 301a-312 may further comprise one or more analog or digital measurement devices, such as flow meters. In an embodiment, flow meters may be operable to measure the flow of inlet wastewater 600a,b and/or effluent or discharged wastewater 700, for example.

As discussed herein, in an embodiment data (electronic signals) representing a measured current or voltage (i.e., measurements) and a sampled wastewater flow, for example, may be communicated or sent to the computation component 500 (e.g., one or more controllers) from a measurement component 301a-312 (e.g., sensors, one or more flow meters) via communications component 400 and path 450.

Upon reception of the data, the computation component 500 may be operable to (i) convert the data representing the measured current or voltage measurements into a BOD concentration, for example, and compute a BOD load based on the computed BOD concentration and a sampled wastewater flow, or (ii) convert the data representing a measured current (or voltage) into BOD concentration data and compute a real-time or near real-time BOD load based on the so-converted data and wastewater flow data.

It should be understood that a BOD sensor is only one example of the type of sensors that may comprise measurement components 301a-312. In additional embodiments the components 301a-312 may comprise one or more nitrogen sensors, and/or one or more sensors for measuring TSS, and/or one or more sensors for measuring a constituent or parameter of interest in wastewater treatment as known to those skilled in the art.

Accordingly, in an embodiment data (electronic signals) representing a measurement completed by such sensors may be communicated to the computation component 500 from a measurement component 301a-312 via communications component 400 and path 450.

Upon reception of such data and data related to a related flow, the computation component 500 may be operable to convert the data and compute one or more real-time or near real-time parameters, such as: nitrate concentrations, nitrate loads, solids loads and removal rates to name just a few of the many computations that may be completed by the computation component 500 based on receipt of such data.

Referring back to FIG. 2, as previously described, but now described in more detail, it is the computational and analysis section 500a that may be operable to complete the computations and analyses based on the data (e.g., signals) received from the measurement component 301a-312 described herein.

In the discussion above we introduced the computation of BOD and COD loads based on measured BOD and COD concentrations and flow data. In yet more detail, upon receiving data from a measurement component 301a-312 (e.g., BOD sensor), the computation and analysis section 500a may be operable to complete a plurality of computations and analyses (collectively “computations” or “computing”), among them: (1) converting a measured current (or voltage) into a BOD concentration; (2) computing a BOD concentration based on a mid-point approximation; (3) computing a BOD concentration based on a period-average approximation; (4) computing a BOD concentration based on a linear-interpolation approximation; (5) computing a BOD concentration based on a “left-hand” approximation; (6) computing a BOD concentration based on a “right-hand” approximation; (7) computing an average, minimum and maximum (i.e., peak), periodic wastewater flow (e.g., instantaneous, daily, weekly, monthly); (8) computing BOD loads based on (2) through (7); and (9) computing interpolated BOD loads.

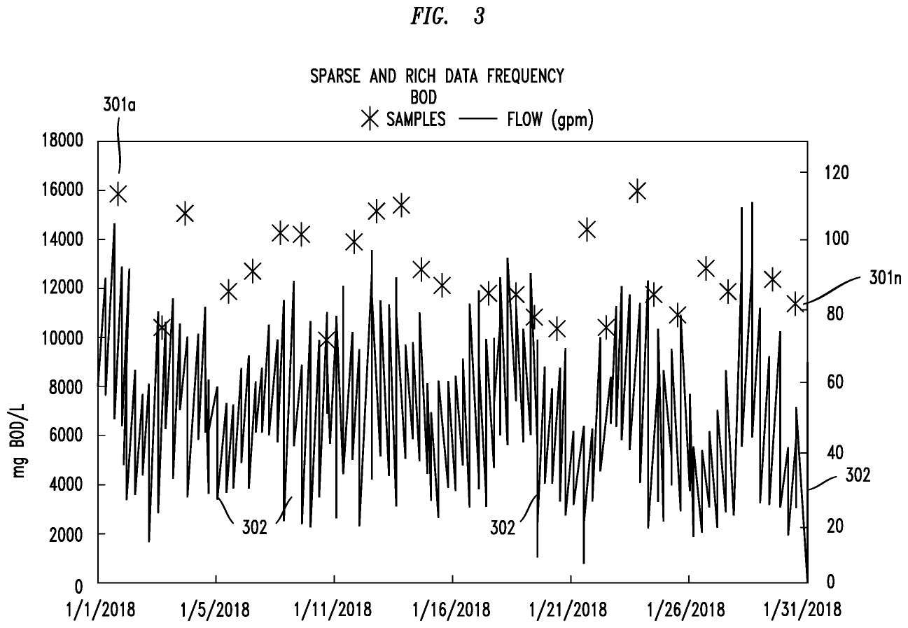

Referring now to FIG. 3 there is depicted an exemplary graph of BOD concentration data 301a-n (i.e., samples) (where “n” is the last sample) measured and sampled in accordance with one or more embodiments of the invention.

In embodiments of the invention, one or more of the measurement components 301a-312 may comprise a PLC operable to sample raw data from one or more sensors (e.g., BOD sensor), flow meters and other measurement components 301a-312 over a selectable length of time, where the time between each sample may be referred to as a sampling interval and the number of raw data samples that are sampled over a selectable time interval may be referred to as a sampling frequency. For example, an exemplary PLC may be operable to sample raw data over a time interval, typically ranging from microseconds to minutes.

In yet another embodiment, a sampling interval may be longer, and therefore the sampling frequency may be lower, such as one sample per hour, per day, per week, or per month.

FIG. 3 also depicts wastewater flow data 302 that may be sampled using an in-line flow meter 301a-312, for example.

In the embodiment depicted in FIG. 3 the wastewater flow data 302 may be sampled instantaneously (e.g., every 1 to 1000 milliseconds using a PLC) while the BOD concentration data 301a-n may be sampled periodically (e.g., every day). Accordingly, it can be seen that BOD concentration may not be sampled as often as the flow is sampled.

In an embodiment, the computation and analysis section 500a may be operable to “normalize” (i.e. adjust, align) the BOD concentration and flow data samples. That is to say, when the sampling interval and/or sampling frequency used to sample BOD concentration data differs from the interval and/or frequency of the measurement/sampling of wastewater flow data, the section 500a may be operable to execute instructions to adjust such data, in effect, generating wastewater flow data that is sampled over the same time period and/or interval as the BOD concentration data or vice-versa. Herein, the process of adjusting one set, or both sets, of data so that the time period over which both sets of data are sampled is the same may be referred to herein as “normalizing” or “normalization” of such data by the section 500a.

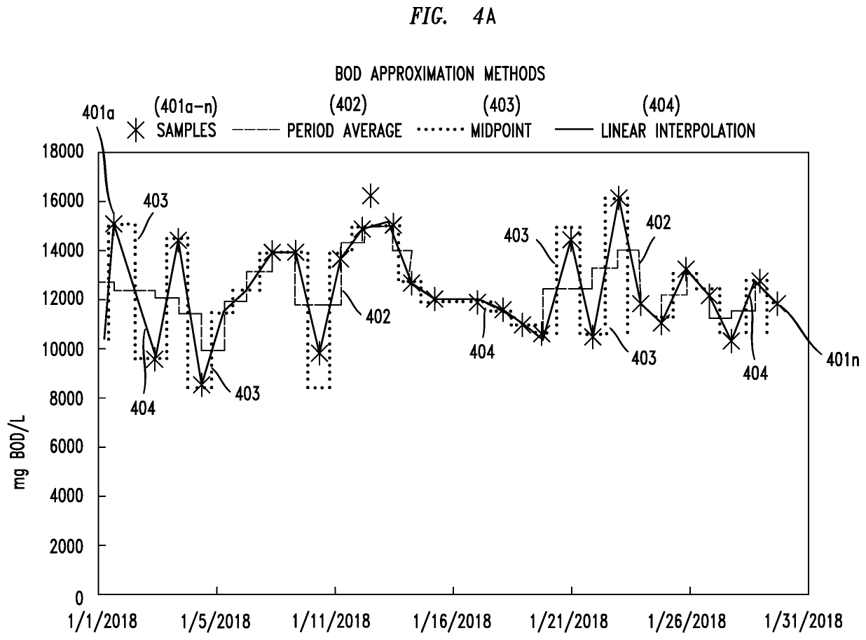

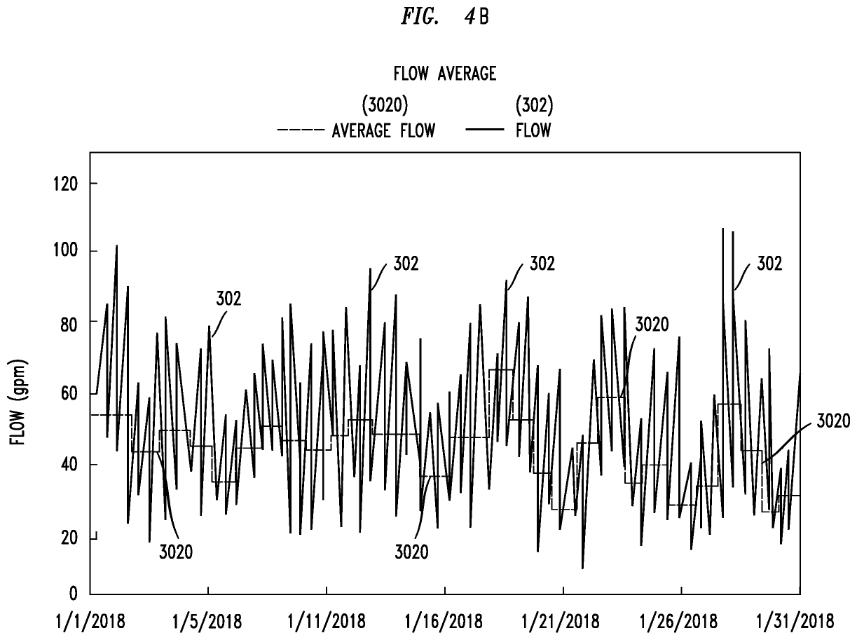

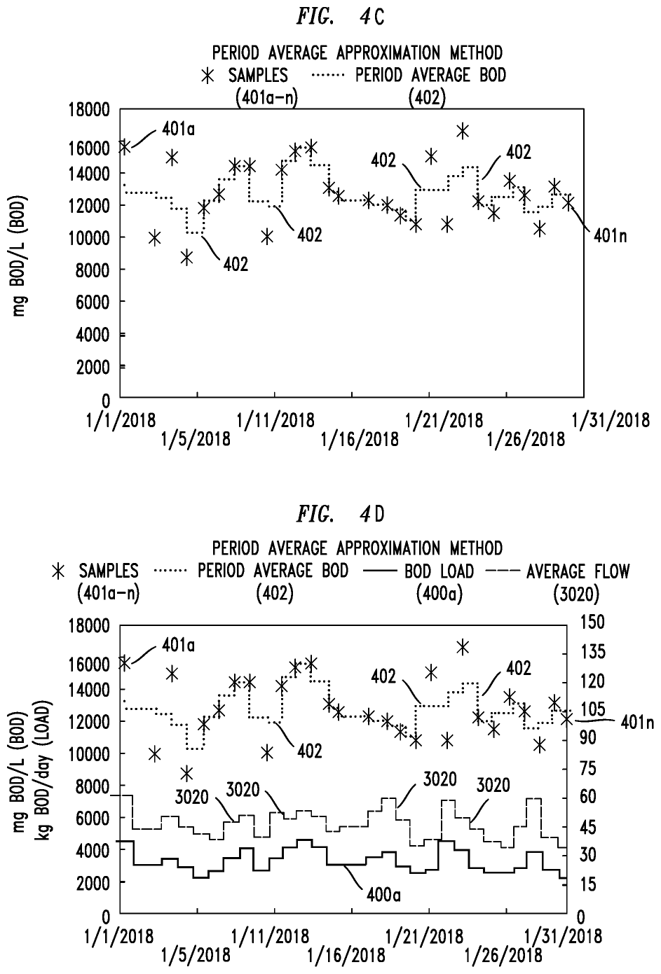

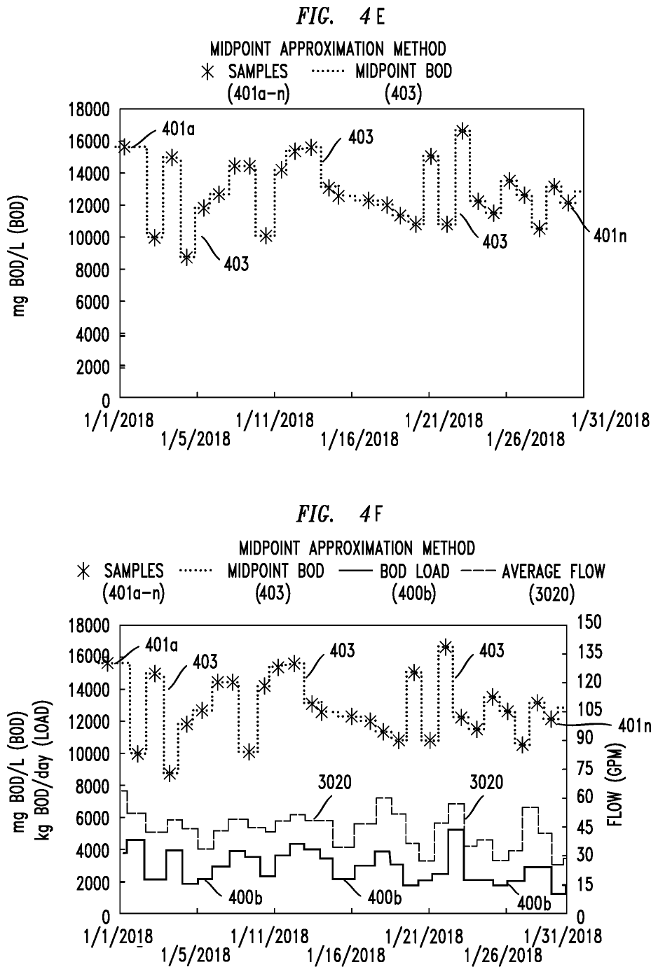

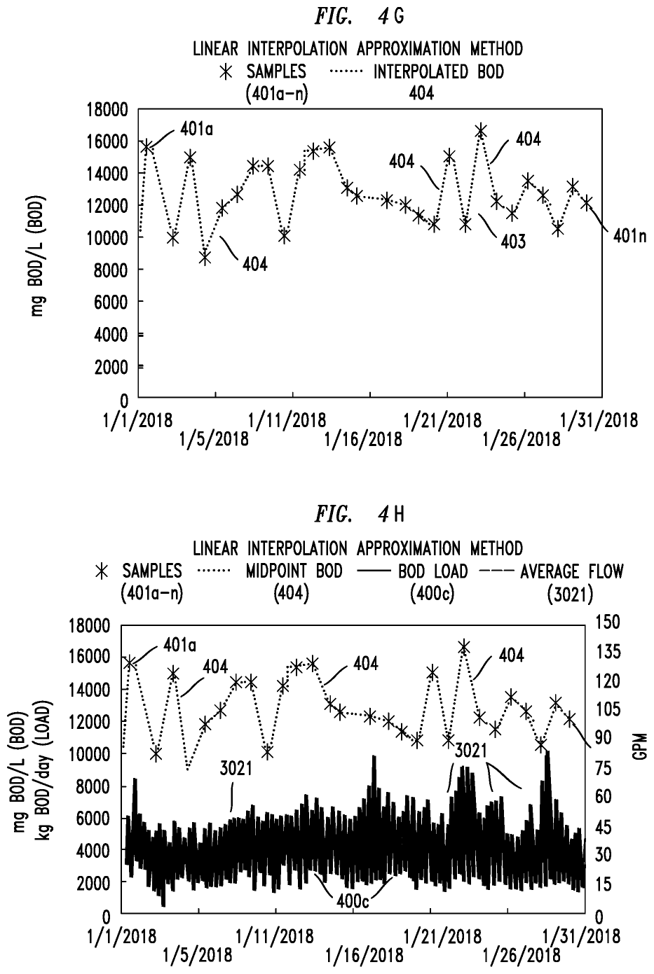

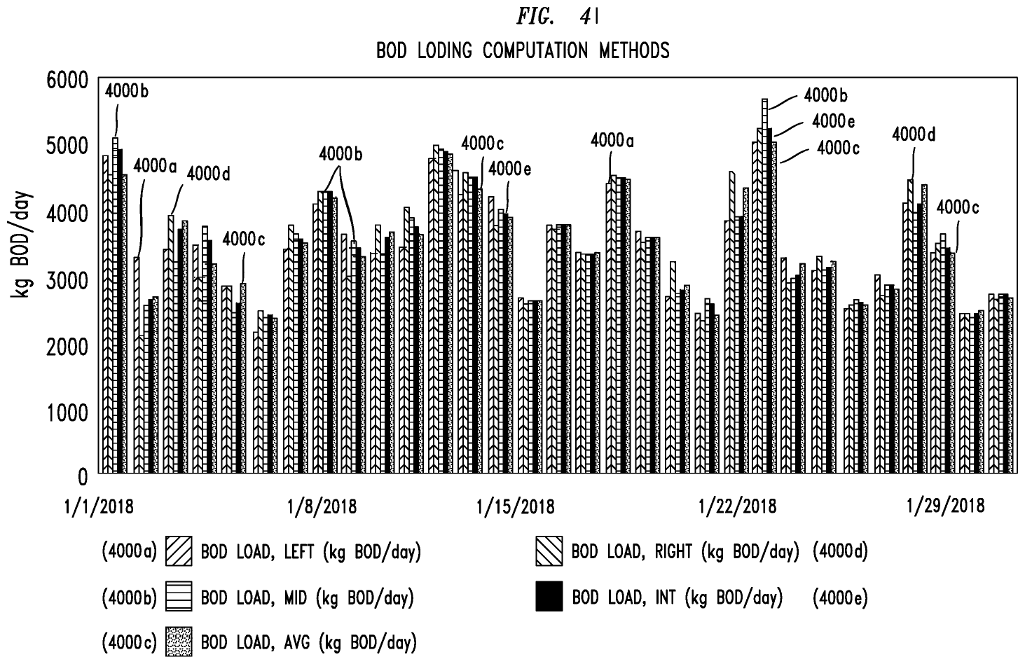

Referring now to FIG. 4A there are depicted graphs of a plurality of methods for computing BOD concentrations from BOD data samples according to embodiments of the invention. As shown, these graphs include graphs generated in accordance with an inventive period average approximation method 402, mid-point approximation method 403, and an inventive linear interpolation approximation method 404.

In FIG. 4A, samples 401a-n (where “n” corresponds to the last data sample) comprise raw data samples associated with a wastewater, BOD concentration. It should be understood that in the embodiments illustrated by the graphs in FIG. 4A, only samples 401a-n are actual, raw data samples. It should be further understood that the three methods illustrated in FIG. 4A are not exhaustive, and that other methods may be used to compute BOD concentrations. For example, an inventive “left-hand” or “right-hand” approximation methods may be used. In an embodiment, in a left-hand approximation method a BOD concentration data sample is assumed to be valid until a “next” data sample is received, while in a right-hand approximation method a sample represents a BOD concentration prior to a “previous” sample. Other methods may also be used, including, but not limited to, those that use inventive spline/smoothing/fitting functions, process dynamics modeling to predict changes in BOD concentrations as a function of other parameters, such as wastewater flow rates and vessel liquid volumes and levels, for example.

Before continuing, it should again be understood that each of the approximation (and data reduction) methods described herein and their equivalents may be completed by a combination of the elements 201a through 500 depicted in FIGS. 1 and 2, and particularly, by the execution of stored electronic instructions by the computation and analysis section 500a in combination with received and stored BOD concentration data (samples) and/or other required data to complete the computations corresponding to a particular method.

In sum, the computation and analysis section 500a may be operable to execute stored instructions to complete each and all of the methods used to generate the graphs in FIG. 4A, to in effect, estimate or approximate the BOD concentration of a wastewater stream, for example, for all times in between samples 401a-n.

In embodiments of the invention, each inventive method has its advantages and disadvantages. For example, an inventive linear approximation method may be a more accurate method to approximate BOD concentration, but may also require more processing cycles by the computation and analysis section 500a when compared to other methods. Conversely, the left or right-hand approximation method may require the fewest processing cycles and thus require a less complex or powerful controller or processor as a part of section 500a. However, such methods may be less accurate. In general then, the specific operating and performance parameters associated with a controller (or PLC, processor or processors) making up the computation and analysis section 500a required to complete a particular method described herein may be determined by the specific application, and/or by the end-user's need for more or less accuracy or more or less complexity, for example.

Before continuing, it should be further noted that each of the physical embodiments of the components depicted in FIGS. 1 and 2, and in particular the computation and analysis section 500a may comprise the necessary electronics to enable each to process data and signals much faster than humanly possible. That is to say, each of the exemplary embodiments of the present invention cannot practically be implemented in any amount of time that would be acceptable to one skilled in the art using human beings as substitutes for the systems, devices, and components described herein. For example, many of the embodiments described herein involve an exchange of data (signals) via a measurement component 301a-312, communications component 400 and a computation component element 500 over a communications path 450 (e.g., communications network), where such components may be remotely located from one another, and where the data exchanged may be required to be available for immediate use by another physical device or component or for display to a user involved in the exchange of data. Accordingly, the speeds at which the data is exchanged, and the amount of data exchanged, is many times faster and greater than that which can be communicated and processed by the human mind. Nor can such data be processed or displayed by the human mind or mechanical means (pen and paper) within the time periods demanded by users of the present invention and those skilled in the art of the present invention.

Referring now to FIG. 4B there is depicted an exemplary graph 3020 of values of an average flow computed, for example, by section 500a based on measured and sampled wastewater flow data 302. In an embodiment of the invention, section 500a may, thereafter, be operable to compute a BOD load by, for example, multiplying the computed BOD concentration values derived using one of the methods depicted by the graphs in FIG. 4A by the computed wastewater flow values 3020 (e.g., average flow).

We now present a more detailed discussion of the use of each of the exemplary BOD concentration approximation methods illustrated in FIG. 4A in conjunction with an exemplary wastewater flow computation method to compute a BOD load.

Referring now to FIG. 4C there is depicted a graph 402 of BOD concentration values computed by the section 500a using an exemplary period-average approximation method based on sampled, raw data 401a-n. In this embodiment, the computation and analysis section 500a may be operable to receive the BOD concentration data samples 401a-n and execute appropriate stored instructions to complete the computations corresponding to an inventive period approximation method in order to estimate additional BOD concentration values falling in between samples 401a-n. Graph 402 represents a visual graph that connects the samples 401a-n and the additional data values.

In this embodiment, the stored instructions executed by the computation and analysis section 500a include instructions that compute averaged values based on samples 401a-n at either end of a time interval (e.g., every second, millisecond, every minute, etc . . . ) because the inventive period average approximation technique assumes that a time period between samples 401a-n can be represented by such an average.

In FIG. 4D, computed BOD concentrations are graphically depicted by graph 402 along with graphically depicted, computed average flows 3020 and computed BOD loads 400a. In an embodiment, the section 500a may be operable to compute the BOD loads 400a by multiplying, for example, the values of the BOD concentrations represented by graph 402 by the values of average wastewater flows represented by graph 3020.

Referring now to FIG. 4E there is depicted an exemplary graph 403 of BOD concentrations computed by the section 500a using an exemplary midpoint approximation method based on sampled, raw data 401a-n. In this embodiment, the computation and analysis section 500a may be operable to receive the BOD concentration samples 401a-n and execute appropriate stored instructions to complete the computations corresponding to a midpoint approximation method in order to estimate additional BOD concentration values. FIG. 4E represents a visual graph 403 that connects the samples 401a-n and the additional values.

In this embodiment, the stored instructions executed by the computation and analysis section 500a include instructions that compute values that are half-way (time-wise) in between each sample 401a-n because the inventive midpoint approximation technique assumes that an instantaneous BOD concentration value is by the nearest (time-wise) sample 401a-n (prior or after a data point 401a-n).

In FIG. 4F, the computed BOD concentrations are graphically depicted by graph 403 along with graphically depicted, computed average wastewater flows 3020 and computed BOD loads 400b. In an embodiment, the section 500a may be operable to compute the BOD loads 400b by multiplying, for example, the values of the BOD concentrations represented by graph 403 by the values of average wastewater flows represented by graph 3020.

Referring now to FIG. 4G there is depicted an exemplary graph 404 of BOD concentrations computed by the section 500a using an exemplary linear interpolation approximation method based on sampled, raw data 401a-n. In this embodiment, the computation and analysis section 500a may be operable to receive the BOD concentration data samples 401a-n and execute appropriate stored instructions to complete the computations corresponding to the inventive linear-interpolation approximation method in order to estimate additional BOD concentration values falling in between samples 401a-n. FIG. 4G represents a visual graph 404 that connects the samples 401a-n and the additional values.

In this embodiment, the stored instructions executed by the computation and analysis section 500a include instructions that compute values that are located at points along a linear line connecting two samples 401a-n because, for example, a linear-interpolation approximation method may assume that an instantaneous BOD concentration value between two samples 401a-n corresponds to a linear change in BOD concentration at a point in time between two samples 401a-n.

In FIG. 4H, the computed BOD concentrations are graphically depicted by graph 404 along with graphically depicted, computed wastewater flows 3021 and computed BOD loads 400c. In an embodiment, the section 500a may be operable to compute the BOD loads 400c by multiplying, for example, the values of the BOD concentrations represented by graph 404 by the values of flow represented by graph 3021.

Referring now to FIG. 4I there are depicted graphical representations 4000a to 4000e of daily BOD load computations computed in accordance with embodiments of the invention by the computation and analysis section 500a, for example.

More particularly, FIG. 4I depicts: (i) bar graphs 4000a, each representing BOD loads computed by section 500a based on combining BOD concentrations computed using inventive left-hand approximations, with wastewater flow computations; (ii) bar graphs 4000b, each representing BOD loads computed by section 500a based on combining BOD concentrations computed using inventive midpoint approximations, with wastewater flow computations, (iii) bar graphs 4000c, each representing BOD loads computed by section 500a based on combining BOD concentrations computed using inventive period average approximations, with wastewater flow computations, (iv) bar graphs 4000d, each graph representing BOD loads computed by section 500a based on combining BOD concentrations computed using inventive right-hand approximations, with wastewater flow computations, and (v) bar graphs 4000e, each representing BOD loads computed by section 500a based on combining BOD concentrations computed using inventive linear interpolation approximations, with wastewater flow computations.

Having presented embodiments that exemplify systems and related methods for (i) measuring, sampling and computing BOD concentrations and wastewater flows, and (ii) computing BOD loads based on such concentrations and flows, we now turn to a discussion of exemplary embodiments that (iii) further analyze and present the results of such analyses in the form of one or more reports to a user so that such a user can further provide water and energy-related services to his or her (or its) end-customers.

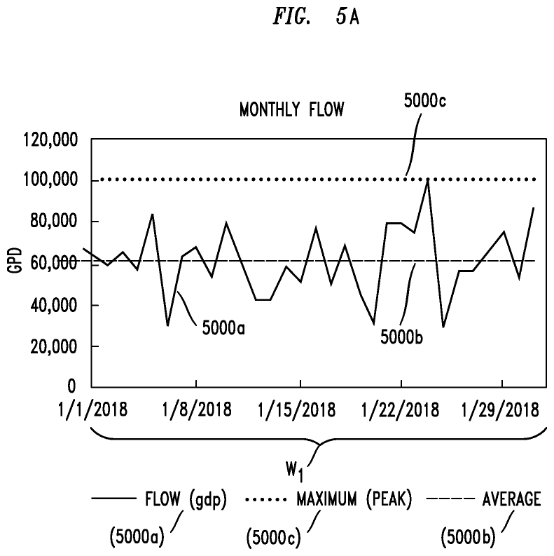

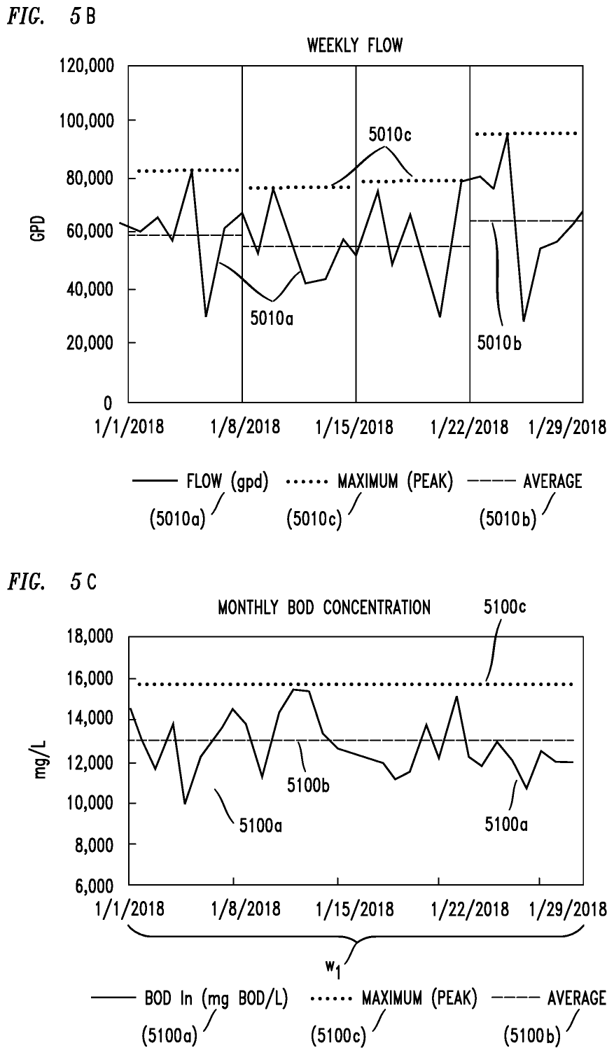

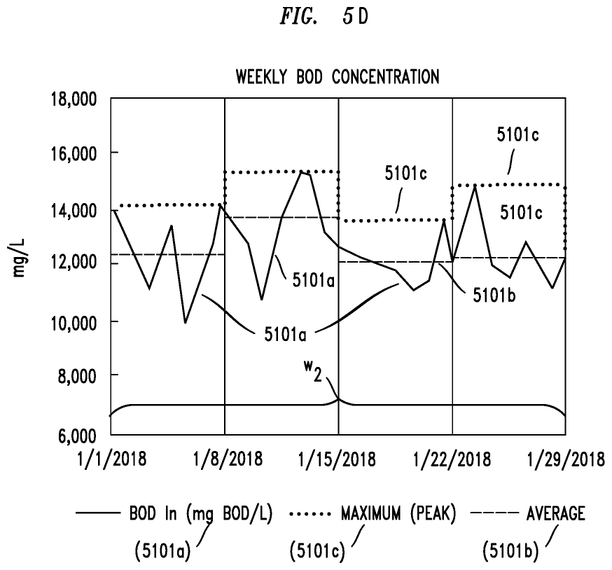

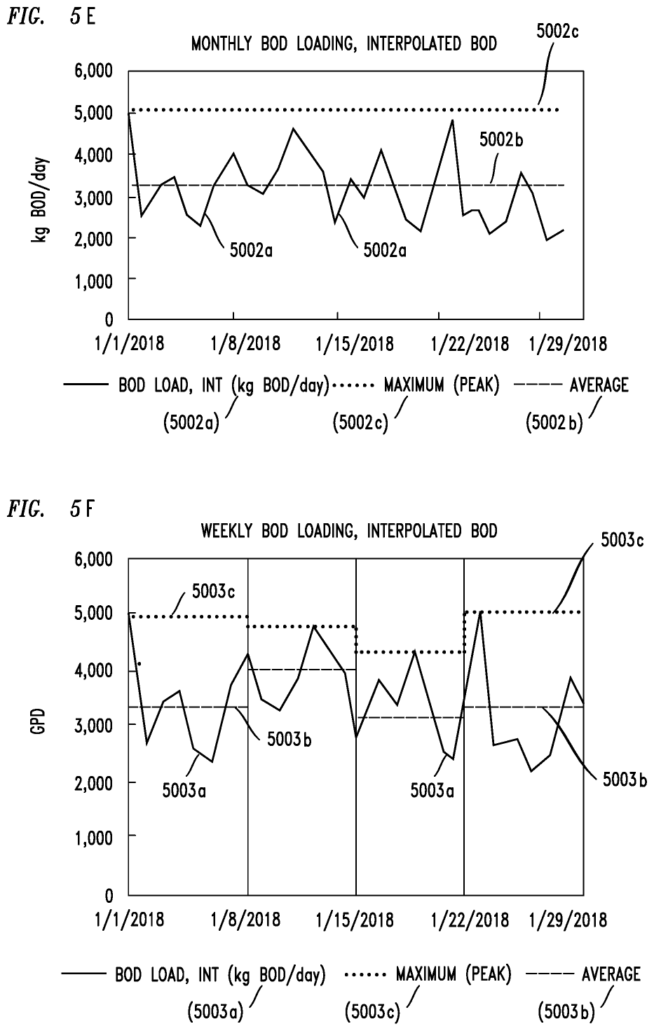

Referring now to FIG. 5A there is depicted exemplary graphical representations 5000a to 5000c of wastewater flow values. More particularly, there are depicted graphs 5000a to 5000c of daily, computed wastewater flow values 5000a, a computed average wastewater flow value 5000b and a computed peak wastewater flow value 5000c that may be generated by section 500a.

It should be understood that the computations and graphs described herein and shown in the drawings are merely exemplary and are presented to help illustrate the concepts and features of the invention. Similar computations, analyses, and graphs may be constructed for additional measured parameters of the system (e.g., water constituents described herein, energy and power, stream volumes, and levels of constituents or loads in streams. Such graphs may be a part of one or more reports that may be generated by components of systems provided by the present invention, for example. These reports may be used by a user of the inventive systems described herein to provide water and energy-related services to his or her (or its) end-customers.

Continuing, in an embodiment the average and peak wastewater flow values 5000b and 5000c, respectively, may be computed by section 500a by analyzing the daily, computed wastewater flow values 5000a. For example, the peak wastewater flow value 5000c may be computed by determining a maximum value of wastewater flow from all of the values represented by graph 5000a during the reporting time period while the average wastewater flow value 5000b may be computed by averaging all of the daily wastewater flow values represented by graph 5000a during the reporting time period, for example.

Further, in an embodiment, each of the exemplary graphs 5000a to 5000c may be generated using a monthly reporting time period or window, w₁, though it should be understood that other time periods or windows may also be used. For example, FIG. 5B depicts the graphical representation of computed daily flow values 5010a, a computed average flow values 5010b and computed peak flow values 5010c based on a weekly reporting time period or window, w₂.

It should be understood that the peak and average flow values represented by graphs 5010b and 5010c may be computed by section 500a by similarly analyzing the daily, computed wastewater flow values 5010a. For example, the peak wastewater flow values 5010c may be computed by determining a maximum value of wastewater flow from each of the values represented by graph 5010a during a weekly reporting time period while the average wastewater flow values 5010b may be computed by averaging each of the wastewater flow values represented by graph 5010a during a similar weekly reporting time period, for example.

In embodiments of the invention, the underlying wastewater flow data used to create the graphs 5000a to 5010c may be measured and sampled by one or more measurement components 301a-312 (e.g., a flow meter) and then transmitted or otherwise sent to the computation and analysis section 500a via component 400 and path 450, for example, so that it may be used to compute BOD loads.

Though not depicted in FIGS. 5A or 5B, it should be understood that a minimum value (or statistical value) of flow values may be similarly computed and graphical represented (e.g. displayed) as well.

Referring now to FIG. 5C there is depicted exemplary graphical representations 5100a to 5100c of computed BOD concentrations (i.e., concentration values). More particularly, there are depicted graphs of daily, computed BOD concentration values 5100a, a computed average BOD concentration value 5100b and a computed peak BOD concentration value 5100c that may be generated by section 500a (e.g., by one or more processors executing stored instructions).

In an embodiment, average and peak BOD concentrations 5100b and 5100c, respectively, may be computed by section 500a by analyzing the daily, computed BOD concentrations 5100a. For example, the peak BOD concentration 5100c may be computed by determining a maximum value of BOD concentration from all of the values represented by graph 5100a during the reporting time period while the average BOD concentration 5100b may be computed by averaging all of the daily BOD concentrations represented by graph 5100a during the reporting time period, for example.

Further, in an embodiment, each of the exemplary graphs 5100a to 5100c may be generated using a monthly reporting time period or window, w₁, though it should be understood that other time periods or windows may also be used. For example, FIG. 5D depicts the graphical representation of computed daily BOD concentration values 5101a, computed average BOD concentrations 5101b and computed peak BOD concentrations 5101c based on a weekly reporting time period or window, w₂.

It should be understood that the peak and average BOD concentrations represented by graphs 5101b and 5101c may be computed by section 500a by similarly analyzing the daily, computed BOD concentration values 5101a. For example, the peak BOD concentration values 5101c may be computed by determining a maximum BOD concentration value from each of the values represented by graph 5101a during a weekly reporting time period while the average BOD concentration 5101b may be computed by averaging each of the BOD concentration values represented by graph 5101a during a similar weekly reporting time period, for example.

In embodiments of the invention, the underlying BOD concentration values used to create the graphs 5100a to 5101c may be measured and sampled by one or more measurement components 301a-312 (e.g., sensors) and then transmitted or otherwise sent to the computation and analysis section 500a via component 400, for example, so that it may be used to compute BOD concentration values.

Though not depicted in FIGS. 5C or 5D, it should be understood that a minimum value (or statistical value) for BOD concentration values may be similarly computed and graphical represented as well.

Having presented exemplary, graphical representations of both flow and BOD concentrations based on monthly and weekly reporting windows, we now present exemplary, graphical representations of BOD loads based on monthly and weekly reporting windows.

In an embodiment, section 500a may compute the data used to generate such graphical representations (and generate the graphs as well) by combining the values used to generate graphs of wastewater flow (5000a to 5010c) and BOD concentrations (5100a to 5101c).

Referring now to FIG. 5E there is depicted exemplary graphical representations 5002a to 5002c of computed BOD loads (i.e., BOD load values). More particularly, there are depicted graphs of daily, computed BOD loads, a computed average BOD load 5002b and a computed peak BOD load 5002c that may be generated by section 500a.

In an embodiment the computed BOD loads in graph 5002a may be computed from BOD approximations using an inventive linear interpolation method, for example.

Further, the average and peak BOD loads 5002b and 5002c, respectively, may be computed by section 500a by analyzing the daily, computed BOD loads 5002a. For example, the peak BOD load 5002c may be computed by determining a maximum BOD load value from all of the values represented by graph 5002a during a reporting time period while the average BOD load 5002b may be computed by averaging all of the daily BOD loads represented by graph 5002a during the reporting time period, for example.

Further, in an embodiment, each of the exemplary graphs 5002a to 5002c may be generated using a monthly reporting time period or window, w₁, though it should be understood that other time periods or windows may also be used. For example, FIG. 5F depicts the graphical representation of computed daily BOD loads 5003a, computed average BOD loads 5003b and computed peak BOD loads 5003c based on a weekly reporting time period or window, w₂.

It should be understood that the peak and average BOD loads represented by graphs 5003b and 5003c may be computed by section 500a by similarly analyzing daily, computed BOD loads 5003a. For example, each of the peak BOD loads 5003c may be computed by determining a maximum BOD load value from all of the values represented by graph 5003a during a weekly reporting time period while each of the average BOD loads 5003b may be computed by averaging all of the BOD load values represented by graph 5003a during a similar weekly reporting time period, for example.

Though not depicted in FIG. 5E or 5F, it should be understood that a minimum value (or statistical value) of BOD load values may be similarly computed and graphical represented (e.g. displayed) as well.

It should be also noted that, although not illustrated by FIGS. 5A-5F, section 500a, for example, may be further operable to complete additional statistical analyses on the data sets (values) and display corresponding graphs. For example, values based on mode, standard deviation, and confidence intervals may be computed. Further, it should be noted that, in addition to BOD concentrations and loads, section 500a may be operable to compute additional values based on additional data measured by the measurement components 301a to 312.

Referring back to FIG. 1, it should be understood that the system 100 may include a display 900 for displaying the graphs in FIGS. 3 through 5F, for example, as one or more reports, for example.

In one embodiment, the component 500 may further comprise a tracking and billing section 500b that may be operable to compute one or more resource-related values or amounts (e.g., fees, rates) based on the computations and analyses completed by section 500a described above and elsewhere herein, which are derived from data samples received from the one or more measurement components 301a-312. In embodiments of the invention, inventive resource-related values or amounts that may be computed by the tracking and billing section 500b may comprise fees derived from initial or start-up of one or more of the treatment components 201a-210 (e.g., fixed costs, such as connection or capacity fees) and fees derived from measurements (data samples) made by one or more of the measurement components 301a to 312 during start-up or during on-going operations (e.g., variable costs, such as costs per volume, mass, or unit energy). In embodiments of the invention, such computations may generate resource-related values or amounts that may be representative of a monetary amount (e.g., a fee), or another valuable consideration amount, or a rate, or may be converted to such an amount or rate, for example. To reiterate what has been mentioned previously, it should be understood that the computations and analyses completed by section 500a are completed much faster than humanly possible. Accordingly, it should be understood that such embodiments of the present invention cannot practically be implemented in any amount of time that would be acceptable to one skilled in the art using human beings as substitutes for the PLCs, controllers, electronic processors or processors that may be used to compete the computations and analyses.

Similar to the computation and analysis section 500a, to complete its computations the tracking and billing section 500b may comprise a controller or processor that is operable to store its own sets of instructions in the form of electrical signals in an on-board or associated memory, store referential, historical and/or statistical data, and retrieve the instructions, any referential, historical or statistical data and the results (graphs, values, amounts) from computation and analysis section 500a in order to complete a particular computation. The instructions may comprise one or more steps or processes that, when combined, make up a particular computation. For example, one set of instructions may form a particular fixed fee computation, while another set of instructions may comprise a particular variable fee computation, for example. The tracking and billing section 500b may be located with or separate from the computation and analysis section 500a and may be operable to access data and/or instructions from the same or separate memory elements 500d.

For example, in one embodiment a fixed fee amount, F_{F}, may be computed based on the total installed capital cost of a system, such as system 100, and/or may depend on the duration of a particular billing period. In an embodiment, F_{F}, may be a constant. Finally, billing factors may be included for a number of parameters or components j (e.g. volumetric throughput capacity, contaminant load capacity, removal capacity) based on the peak capacity factor, F_{P,j}, total billing factor, F_{S,j}, and excess billing factor, F_{E,j}, and may be computed and comprise constants or may be dependent on other factors, such as a time interval, or volume, for example.

In yet another embodiment a fixed fee computation that may be computed by section 500b may be a reserved capacity computation derived from data samples received by (or stored within) the component 500 that is associated with the design and materials comprising one or more treatment components 201a-210 (e.g. the design and structure of an anaerobic treatment component). Other fixed fee computations may be based on one or more stored, constants (e.g., the same data is used for each computation over time) or based on an escalation factor applied to a constant (e.g. a time-dependent multiplier). One example of an escalation factor may be a pre-determined rate or a cost index.

Having presented some exemplary fixed fee computations we now present some exemplary variable computations. Examples of variable computations are those derived from data samples associated with the measurement of (i) the total volume of wastewater treated, which may vary from measurement to measurement, (ii) the volume of wastewater treated over a set time period (i.e., flow rate), which may also vary, (iii) discharge requirements (i.e., measured characteristics of the water being discharged, and/or degree to which a material or mineral has been or must be removed from wastewater), (iv) the measured amount of material (contaminants), such as BOD or COD, initially contained in wastewater loaded into the system 100, or removed from such wastewater by one or more treatment components 201a-210, or those derived from (v) a proxy measurement, e.g., from an in-line sensor or from an electrode that is part of an anaerobic treatment system described in the '763, '896 and '132 Applications operable to function as proxy for measuring BOD based on biologically-catalyzed reactions, for example.

Alternatively, rather than complete computations derived from periodic measurements of the volume of wastewater treated, and/or volume of wastewater treated over a set time period, the computations may be based on pre-determined or stored measurement values.

In an embodiment, section 500b may be operable to compute a variable fee computation from a plurality of measurements (data samples) made by one or more of the measurement components 301a-312 and a plurality of related computations made by the computation and analyses section 500a. For example, one variable fee computation may be derived from one or more sub-computations, such as: a measurement of a treated amount of wastewater and its related computation, a measurement of a treated amount of organic solid waste and its related computation, a measurement of reuse water supplied and its related computation, a measurement of gross or net electricity usage and its related computation, a measurement of gross or net thermal energy usage and its related computation, and a sustainability credit computation, for example.

In embodiments, a variable billing value “i” associated with a measured parameter selected from a list of a plurality of measured parameters, such as: volume, BOD, COD, TN, TS, TSS, VSS, TDS, TKN, ammonia, ammonium, nitrate, nitrite, total phosphate, orthophosphate, total sulfur, sulfate, sulfite, sulfide, pH, temperature, conductivity, or other parameters/measurements. Alternatively, or in addition, changes in the concentrations of the above substances/minerals (i.e., constituents) may be measured and/or computed and used to compute a variable billing rate. Further, the loads or removal rates (concentrations or changes in concentration multiplied by flow rates), may be used for computation.

Accordingly, in one embodiment a rate of one or more parameters i entering a wastewater treatment system over a plurality of different time intervals may be measured, where the rate may be selected from a peak rate or an average rate of the one or more parameters, for example.

Additionally, an overage value for a parameter i may be computed. For example, a value for a parameter i may be computed and then the computed value may be compared to a threshold applicable to the parameter i. When the comparison indicates the computed value exceeds the threshold a difference between the computed value and the threshold may then be determined, where the difference is an overage value.

In another embodiment, the maximum rate or peak rate (the two phrases may be used interchangeably herein) of a measured parameter i entering the system 100, Pᵢ, may be calculated or computed (the two phrases may be used interchangeably herein) over a plurality of different time intervals (e.g., peak minute, peak hour, peak day, peak week), and associated with a billing cycle.

In an embodiment, the peak rate Pᵢ may be computed by section 500b based on a maximum of the concentration of component i (C_{i,t}) (i.e., the highest, validated measurement or computation during a specified time period) or as a loading rate of component i (L_{i,t}) in which the concentration, C_{i,t}, may be multiplied by the flowrate of a stream (Qₜ) over a specific time interval (t), in which case the time intervals used in computing the concentration must match the time interval used in computing a flowrate of the stream (e.g., same instantaneous reading, same minute, same hour, etc.).

The totalized amount of component i, Sᵢ, may be computed by section 500b based on the total amount of the component (Cᵢ) loaded into, or removed from, the system 100. In an embodiment, the amount removed may be computed by generating the sum of the product of the total volumetric flow through the system, (V), and the difference in the inlet, (C_{i,t,1}), and outlet, (C_{i,t,2}), concentrations over all time intervals for a given time interval t, represented as follows:

(Loading) Sᵢ=ΣₜC_{i,t,1}×Vₜ

(Removal) Sᵢ=Σₜ(C_{i,t,1}−C_{i,t,2})×Vₜ

Further, the excess (overage) amount, Eᵢ, for component i may be computed by section 500b by first determining whether Sᵢ exceeds a threshold (Mᵢ) and, if it does, then computing the difference:

If: Sᵢ≥Mᵢ Then: Eᵢ=Sᵢ−Mᵢ Else: Eᵢ=0

Yet further, a percent removal nᵢ of parameter i may be computed from an influent stream of the wastewater by the treatment system, where the percent removal is computed from an average inlet concentration, (C_{i,1},), and an average outlet concentration, (C_{i,2}), where

$${n}_{i} = \frac{{c}_{i , 1} - {c}_{i , 2}}{{c}_{i , 1}} \times 100 \% .$$

Still further, the tracking and billing section 500b may be operable to associate a stored, pre-determined resource-related value (e.g., monetary amount) to a computation generated by section 500a, value or indicator described herein. Pre-determined resource-related values may comprise a variety of stored values, such as: (1) a value based on “net energy billing” (e.g., differential rates for the supply of energy to, and generation of energy by, the system 100 to account for incentives and a premium on renewable energy); (2) a value based on incentives to install and operate an energy and water efficient and renewable-oriented system, such as system 100; (3) a value based on water reuse incentives; (4) end-user specific values; (5) local water and energy-specific resource values; (6) peak usage data; and (7) the difference between the value of a given measured characteristic of inlet wastewater versus the value of the same measured characteristic of discharged water, for example.

In embodiments, the computations based on peak usage data, among other computations, may be used when “on demand” features are desired and in effect (i.e., become operational).

The tracking and billing section 500b may be further operable to categorize or otherwise group a value, amount or indicator generated by the computation and analysis section 500a into one or more resource-related categories. For example, computations having values within a first range, or not exceeding a first value or amount may be associated with a first category, while computations having values within a second range, or not exceeding a second value or amount may be associated with a second category. There may be a plurality of such categories (i.e., more than two). Examples of such resource-related categories may be “premium”, “standard” and/or “discounted”, for example. Yet further, each of these categories may be associated with a monetary value associated with a cost associated with a given computation(s) and their underlying measurements.

Other examples of categories are those based on a particular measurement. For example, computations based on measurements of discharged water 700 from system 100, for example, may be categorized into one or more different categories based on the presence, absence or amount of certain harmful or undesirable minerals, substances or material (i.e., constituents) in the discharged water 700. One category may be associated with “roughed” wastewater which is suitable for discharge to a treatment facility (publicly owned treatment works [POTW] or an offsite disposal facility). Such a category may be associated with a particular fee or rate (i.e., a higher or lower rate than water that is not roughed). Additional exemplary categories (and thus fee amounts or rates) may be associated with the measured properties of the inlet 600a, 600b or discharged water 700, such as the extent to which such water contains an amount of one or more of the following: COD, BOD, TS, TSS, VSS, TDS, TN, TKN, ammonium, nitrate, nitrite, orthophosphate, total phosphorus, sulfate, total sulfur, or metal ions. Still other categories may be based on other measurement parameters such as pH, temperature, conductivity.

It should be understood that one or more of the computations described herein may be a “proxy” measurement for an actual measurement (i.e. a measured value is determined by correlation). For example, an electrode that is part of an anaerobic treatment system described in the '763, '896, '264 or '132 Applications may be operable to function as proxy for measuring a BOD concentration based on reactions of microbes with the electrodes. Likewise, a bio-electrochemical sensor like one set forth in the '082 Application may be used to determine concentrations of other components such as nitrate or nitrite. For example, one or more of the measurement components 301a-312 (in particular 303) may be operable to measure an amount of methane produced as a proxy for the measurement of an amount of BOD that has been treated. In turn, this or other measurements may be used as a proxy to the BOD loading of the system. Alternatively, a component 301a-312 may be operable to indicate BOD loading based on BOD mass flow (mass per time) measurements.

In an embodiment, one or more of the treatment components 201a-210 may comprise one of the systems, devices and apparatuses described in the 763, '132, '149, '401, '516, '212, '462, '896, '801, '161 and '722 Applications, for example. Such treatment components 201a-210 may generate energy directly or indirectly. For example, one or more of the treatment components 201a-210 (e.g., 204) may generate a biogas (e.g. methane or hydrogen) or electricity (e.g., direct current) when one or more of the treatment components 201a-210 comprise a microbial fuel cell, for example. In an embodiment, one or more of the measurement components 301a-312 may comprise one of the systems, devices and apparatuses described in the '401, '817, '264, and '082 Applications. For example, one or more of the measurement components 301a-312 (e.g. 301a, 301b, 306, 310, 311, and 312) may comprise a sensor for measuring a proxy of BOD or a sensor for measuring nitrate.

Further, biogas that is generated may be combusted in an engine or turbine (not shown in figures) to produce electricity, fired in a boiler or a flare to produce heat, or oxidized to capture heat and electricity using a fuel cell. Each of the various energy types may be interconverted as needed. For instance, electricity can be produced from heat using a variety of devices/processes (e.g. heat engine, Carnot engine, Stirling engine, thermoelectric junction—not shown in figures). Energy generated by one or more treatment components 201a-210 may also be captured in the form of chilling capacity (e.g. using an adsorption chiller—not shown in figures).

Thermal energy recovered and generated from the treatment of wastewater by one or more of the treatment components 201a-210 may be captured and transferred to a separate medium (e.g. using a heat exchanger, not shown in figures) for storage and/or used directly. Thermal energy that is generated by one or more of the treatment components 201a-210 may be used by an end-user of the system 100 by incorporating additional open-loop or closed-loop structures (not shown in figures) that use a type of coolant, such as water, air, glycol, or a refrigerant.

Although energy conversion apparatus (e.g. combustion technology for cleaning, and further utilizing methane, electrical generators, heaters, heat exchangers, chillers), and associated support devices, are not shown explicitly in the drawings, it should be understood that such apparatuses and their corresponding functions and processes can be substituted or added to an inventive system at an input, output, and/or within inventive system 100 and may communicate to and from the computation and analysis section 500, for example.

In yet additional embodiments, additional measurement components located outside of the treatment system 100 (not shown in the figures, e.g., located at other parts of a user's facility), may also be included and may communicate with (to and from) the computation and analysis section 500. For example, communications exchanged with the computation and analysis section 500 may be used to control the operation of equipment outside of the system 100. More particularly, in one embodiment one or more flow meters that are not a part of system 100 may communicate with section 500 to control the flow of water of other elements of a user's facility. Still further, data stored or originating at such other elements or from third party sources (e.g., brewery related data, weather related data) may be exchanged with section 500 and, thereafter, utilized to control the system 100, for example.

In accordance with embodiments of the invention, the computation and analysis section 500a may be further operable to compute a value of a net energy balance in a number of ways. For example, the section 500a may be operable to compute a value of a net energy balance by deducting the energy generated by the system 100 from energy used by the system 100.

Alternatively, the section 500a may be operable to separately compute the energy or power used from the energy or power generated and assign different values to each computation.

After section 500a has made the exemplary computations, the tracking and billing section 500b (e.g., specialized microcomputer, controller, processor) may be operable to assign each computed value a resource-related value (e.g., fee or rate). In embodiments of the invention, different resource-related values may be assigned to each computed value.

The tracking and billing section 500b may be operable to compute one or more resource-related values or amounts (e.g., fees, rates) based on the amount of a material (e.g., a biogas, such as methane, a biosolid or sludge) generated as a by-product of treating wastewater (see positions 800 to 806 in FIG. 1) that is measured by a measurement component 302-309, for example, and computed and analyzed by section 500a.

Yet further, tracking and billing section 500b may be operable to compute one or more resource-related values or amounts (e.g., fees, rates) based on a combination of computations and their related measurements/analyses, such as a value that is based on computations that involve a combination of the amount of energy or power used or generated with an amount of material that is generated as a by-product of treating wastewater (e.g., a biogas, such as methane, a biosolid or sludge). In some instances, the resource-related value assigned to a computed value may be zero, indicating, for example, that no fee is due with respect to energy generated. For instance, the value of energy produced and/or consumed by the water treatment system may be excluded.

In more detail, the resource-related value assigned to one or more of the parameters used in the computation of a peak rate of one or more measured parameters, such as F_{F} (fixed fee amount), F_{P,i} (peaking billing factor for a measured component i), F_{S,i} (a totalizing billing factor for component i), F_{E,I} (excess billing factor for component i) may be zero.

In additional embodiments, the tracking and billing section 500b may be operable to assign an incremental, resource-related value to a computed energy value. For example, when section 500a computes a value based on measurements of electrical energy, the resource-related value assigned to such a computed value may represent kilowatt-hours (kWh). Similarly, when section 500a computes a value based on measurements of thermal energy, the resource-related value assigned to such a computed value may represent British thermal units (Btu) or Therm.

Tables 1 through 4 below depict data (e.g., stored, historical data that may be supplied by a source outside of system 100) and resource computation values used to generate a final fee (i.e., bill, invoice) that may be generated by the tracking and billing section 500b according to one embodiment. For example, Table 1 depicts a summary or report of estimated resource-related values that may be provided by a regulated utility, for example, to an end user of system 100. That is, these values may not be generated by section 500b, but may be supplied to the system 100 by a utility. These values may be stored in memory associated with, or a part of, section 500b for example, once received. The values set forth in Table 1 may represent flows associated with water consumption and wastewater volume. These values may be used in place of flow meter readings to compute a resource-related value.

TABLE 1
Utility Flow Meter Readings

| Meter Reading | Jul. 20, 2019-Aug. 21, 2019 | | | |
|---|---|---|---|---|
| Meter | Meter | Previous | Current | Con- |
| Number | Size | Reading | Reading | sumption |
| [symbol] 070168996 | 4 | 264.47 | 2027.45 | 1762.98 |
| [symbol] 070168996 | 4 | 4225.32 | 4710.11 | 484.79 |
| Total Consumption | | | | 2,248 |
| in units of water | | | | |
| Total Consumption | | | | 1,681,332 |
| in gallons of water | | | | |

In additional embodiments, the estimated resource-related values may be BOD, COD, TSS, and Oil & Grease discharged to a sewer, to name just a few of the many values.

In an embodiment, one or more of the estimated values in Table 1 (e.g., “Total Consumption In Gallons Of Water”) may be used in combination with (a) historical, stored wastewater ratios and water quality values (see Table 2) and (b) the total amount of monthly recycled water typically used by the user as measured, for example, by aggregating monthly flow data from an online magnetic flow meter, for example, in the permeate line of a reverse osmosis system to compute fees or amounts that would have been paid by an end user if treatment components of an inventive system are not used (“business-as usual, “BAU”, “pre-system operation” or “pre-system” for short; see Table 3 below).

TABLE 2
Historical Values

| Parameter | Units | Basis Value |
|---|---|---|
| Wastewater to Water Ratio | — | 0.68 |
| Total Oil & Grease Concentration | mg/L | 139 |
| TSS Concentration | mg/L | 705 |
| COD Concentration | mg/L | 10,064 |

For example, a total pre-system volume of water may be computed by the section 500a or section 500b. The total pre-system volume of water (line “c” in Table 3 below) may be computed by adding a total consumption in gallons of water (line “a” in Table 3 below) to a “Monthly Recycled Water Produced” (line “b” in Table 3 below), where each of these values may vary on a periodic basis (e.g., monthly).

FIG. 3: Computation Of Pre-System Values & Fees
BAU Bill

| | | Total | Total | Cost | Water |
|---|---|---|---|---|---|
| | Water Bill | (gallons) | (units) | ($/unit) | Bill ($) |
| a. | Water Volume | 1,681,332 | 2,248 | | |
| | From SFPUC Bill | | | | |
| b. | Monthly Recycled | 383,250 | 512 | | |
| | Water Produced | | | | |
| c. | Total BAU Water | 2,064,582 | 2,760 | $8.48 | $23,407 |
| | Volume (a. + b.) | | | | |
| | | Average | Average | Cost | Cost |
| | Sewer Bill | (mg/L) | (lbs/unit) | ($/lb) | ($/unit) |
| | Total Oil & Grease | 139 | 0.86 | $1.17 | $1.01 |
| | Total Suspended Solids | 705 | 4.39 | $1.12 | $4.90 |
| | COD | 10,064 | 62.7 | $0.59 | $37.10 |
| | Flow | | | | $8.28 |
| | Sewer Service Rate | | | | $51.29 |
| d. | Total Sewer Bill | 1,403,916 | 1,877 | | $96,257 |
| | (68% Flow Factor) | | | | |
| e. | Total Cost of Water | | | | $119,664 |
| | BAU Bill (c. + d.) | | | | |

The computed, total pre-system volume of water (line “c” in Table 3) may be adjusted by an established or historical water-to-wastewater ratio (e.g. from Table 2) to determine other pre-system values from a pre-system sewer flow (e.g., line “d” in Table 3) which also may be computed by the section 500a, 500b or another element to generate a pre-system total cost of water (line “e” in Table 3).

Systems, like system 100, provided by the present invention may be configured to decrease the pre-system total cost of water by a substantial amount. In an embodiment, such a decrease may be computed by subtracting the value of the “Total Consumption In Gallons Of Water” from Table 1 multiplied by a monetary amount (e.g., dollar amount) from the pre-system total cost of water value in Table 3, for example. Thereafter, in an embodiment of the invention a net resource-related savings value based on a percentage of the decrease may be computed by sections 500a, 500b by multiplying the value of the decrease by a variable multiplier (e.g., 70%), as illustrated in Table 4:

TABLE 4
Computation Of Resource-Related Savings Value Based
On Decrease In Pre-System Total Cost of Water

| | | |
|---|---|---|
| e. | Total Cost of Water BAU Bill (c. + d.) | $119,664 |
| f. | Total Current Charges (SFPUC Bill) | $49,398 |
| g. | Decrease in Total Water Bill (e. − f.) | $70,266 |
| h. | Final Fee (g. * 70%) | $49,186 |

Alternatively, the resource-related unit value of treatment of contaminants may be defined by a rate structure from a public utility (Table 3) or a fixed fraction thereof (e.g. 70%).

In an embodiment, systems provided by the present invention (e.g., system 100) may repeatedly complete the computations illustrated in Tables 1 to 4 on a periodic basis (e.g., every month). Further, the computations may be stored and, thereafter, used by sections 500a, 500b to compute an average of the previous periodic computations (e.g., compute an average based on the previous twelve months computations). The computed average may be used as “base” or reference value for further, future computations. In an embodiment, the base value may be varied (e.g., increased) based on a pre-determined schedule (e.g., rate schedule), for example. One or more of the tables described herein may form a report that may be provided to an end customer of a user of systems provided by the invention.

From the foregoing description, one skilled in the art can easily ascertain the essential characteristics of the invention and, without departing from the spirit and scope thereof, can make various changes and modifications of the invention to adapt it to various usages and conditions.

We claim:

1. A method for monitoring wastewater comprising:
receiving one or more measurements from one or more sensors or analytical instruments and one or more flow meters over a selectable length of time at a controller, and
computing a contaminant load of the wastewater based on the received measurements.

2. The method as in claim 1 where the controller comprises a programmable logic controller.

3. The method as in claim 1 further comprising:
sampling the measurements from the one or more flow meters instantaneously; and
sampling the measurements from the one or more sensors or analytical instruments periodically, where the sensors and or analytical instruments are not sampled as often as the flow meters during a same time period.

4. The method as in claim 1, where the contaminant load comprises BOD, COD, pBOD (BOD proxy), or pCOD (COD proxy).

5. The method as in claim 4, wherein the one or more sensors comprise one or more BOD sensors for completing pBOD proxy measurements, the one or more BOD sensors comprising one or more electrodes of a bio-electrochemical system, the method further comprising;
measuring an electrical current or voltage generated by the one or more electrodes;
communicating electronic signals representing the measured current or voltage measurements and a sampled wastewater flow from the one or more flow meters to the controller or another controller;
converting the electronic signals representing the measured current or voltage measurements into a BOD concentration; and
computing the BOD load based on the computed BOD concentration and the sampled wastewater flow.

6. The method as in claim 3 further comprising:
adjusting data from the sampling of the one or more flow meters and data from the sampling of the one or more sensors or analytical instruments so that the time period over which both sets of data are sampled is the same.

7. The method as in claim 1 further comprising:
computing one or more resource-related values or amounts derived from the measurements received from the one or more flow meters and from the one or more sensors or analytical instruments.

8. The method as in claim 1 further comprising receiving the measurements from the one or more sensors, analytical instruments and the one or more flow meters during start-up of a wastewater system.

9. The method as in claim 1 further comprising receiving the measurements from the one or more sensors, analytical instruments and the one or more flow meters during operation of a wastewater system.

10. The method as in claim 7 wherein the resource-related values or amounts represent a billing rate.

11. The method as in claim 7 wherein the resource-related values or amounts represent a combination of fixed or variable value derived from measurements received from the one or more BOD sensors and one or more flow meters or analytical instruments.

12. The method as in claim 1 further comprising at least one of the following:
(i) treating a volume of the wastewater over a set time period, and
(ii) measuring an amount of contaminants contained in influent wastewater based on the measurements from the one or more sensors, analytical instruments or flow meters, measuring an amount of contaminant in effluent wastewater based on the measurements from the one or more sensors, analytical instruments or flow meters, and computing an amount of contaminant removed from the wastewater from the measured amount in the influent, the measured amount in the effluent, and a flow rate.

13. The method as in claim 7 further comprising computing one or more resource-related values or amounts derived from data samples associated with one or more of (i) measuring a total volume of the wastewater treated, (ii) measuring a volume of the wastewater treated over a set time period, (iii) characteristics of the wastewater being treated, including, but not limited to, the amount of a contaminant in influent wastewater, (iv) characteristics of the wastewater being discharged, including, but not limited to, the amount of a contaminant in effluent wastewater, (v) a computation of the amount of contaminants removed from the wastewater or (vi) a direct measurement of a current or voltage of one or more biologically-catalyzed electrodes.

14. A method for monitoring wastewater comprising measuring a rate of one or more parameters i entering a wastewater treatment system over a plurality of different time intervals.

15. The method as in claim 14 wherein the rate is selected from a peak rate or an average rate of the one or more parameters.

16. The method as in claim 14 further comprising:
computing a value for a parameter i;
comparing the computed value to a threshold for the parameter i; and
computing an overage value for the parameter i when the comparison indicates the computed value exceeds the threshold by determining a difference between the computed value and the threshold.

17. The method as in claim 14 further comprising computing a percent removal nᵢ of parameter i from an influent stream of the wastewater by the treatment system, where the percent removal is computed from an average inlet concentration, (C_{i,1},), and an average outlet concentration, (C_{i,2}), where
$${n}_{i} = \frac{{c}_{i , 1} - {c}_{i , 2}}{{c}_{i , 1}} \times 100 \% .$$

18. The method as in claim 14 further comprising computing the amount of parameter i loaded into, or removed from, the wastewater by generating a sum of the product of a total volumetric flow of wastewater, (V), and a difference in an inlet concentration, (C_{i,t,1}), and an outlet concentration (C_{i,t,2}), over all time intervals for a given time interval t, represented as follows:
(Loading) Sᵢ=ΣₜC_{i,t,1}×Vₜ
(Removal) Sᵢ=Σₜ(C_{i,t,1}−C_{i,t,2})×Vₜ

19. A method comprising:
collecting data from a wastewater stream associated with one or more parameters i, where the one or more parameters i are measured in an influent or effluent stream of the wastewater stream;
collecting the data over a plurality of different time intervals; and
determining one or more of (i) a peak value of the one or more parameters, (ii) average value of the one or more parameters, and (iii) a total value of the one or more parameters.

20. The method as in claim 19 further comprising associating a stored, pre-determined resource-related value to one or more computations of the one or more parameters i entering the wastewater system over the plurality of different time intervals.
